SEQUENCE LISTING (15 sequences)

SEQ ID NO: 1 - protein, 10 aa, Artificial Sequence
  Description of Artificial Sequence Synthetic peptide
LYNIIKNREG

SEQ ID NO: 2 - protein, 9 aa, Artificial Sequence
  Description of Artificial Sequence Synthetic peptide
YNIIKNREG

SEQ ID NO: 3 - protein, 9 aa, Artificial Sequence
  Description of Artificial Sequence Synthetic peptide
LYNIIKNRE

SEQ ID NO: 4 - protein, 5 aa, Artificial Sequence
  Description of Artificial Sequence Synthetic peptide
SSADL

SEQ ID NO: 5 - protein, 6 aa, Artificial Sequence
  Description of Artificial Sequence Synthetic peptide
LVPRGS

SEQ ID NO: 6 - protein, 4 aa, Artificial Sequence
  Description of Artificial Sequence Synthetic peptide
HFFK

SEQ ID NO: 7 - protein, 10 aa, Artificial Sequence
  Description of Artificial Sequence Synthetic peptide
VVHFFKNIVT

SEQ ID NO: 8 - protein, 5 aa, Artificial Sequence
  Description of Artificial Sequence Synthetic peptide
VHFFK

SEQ ID NO: 9 - protein, 9 aa, Artificial Sequence
  Description of Artificial Sequence Synthetic peptide
LIAFTSEHS

SEQ ID NO: 10 - protein, 9 aa, Artificial Sequence
  Description of Artificial Sequence Synthetic peptide
FTSEHSHFS

SEQ ID NO: 11 - protein, 11 aa, Artificial Sequence
  Description of Artificial Sequence Synthetic peptide
FFRMVISNPAA

SEQ ID NO: 12 - protein, 9 aa, Artificial Sequence
  Description of Artificial Sequence Synthetic peptide
FRMVISNPA

SEQ ID NO: 13 - protein, 11 aa, Artificial Sequence
  Description of Artificial Sequence Synthetic peptide
SLRTLEDNEER

SEQ ID NO: 14 - protein, 21 aa, Mus musculus
MEVGWYRSPFSRVVHLYRNGK

SEQ ID NO: 15 - protein, 21 aa, Homo sapiens
MEVGWYRPPFSRVVHLYRNGK

(19) United States
(12) Patent Application Publication
(10) Pub. No.: US 2010/0279401 A1
(43) Pub. Date: Nov. 4, 2010

(54) T Cell Hybridomas And Related Compositions And Methods For Assaying And Modulating T Cell Receptor-Mediated Immune Responses

(76) Inventors: Gregory G. Burrows, Portland, OR (US); Yuan K. Chou, Portland, OR (US); Arthur A. Vandenbark, Portland, OR (US)

Correspondence Address: Jeffrey J. King, Esq.;Patent Networks Law Group PLLC, 5000 Carillon Point, Suite 400, Kirkland, WA, 98033, US

(21) Appl. No.: 12/527,004

(22) PCT Filed: Jul. 7, 2005
(86) PCT No.: PCT/US05/24320

§ 371 Date: Jul. 2, 2010

Related U.S. Application Data
(60) Provisional application No. 60/586,433, filed on Jul. 7, 2004.

(51) Int. Cl. C12N 5/16

(52) U.S. Cl. 435/346

(57) ABSTRACT

The present invention provides T cell hybridomas and related compositions and assay systems for investigative, diagnostic, and therapeutic use in modulating T cell receptor (TCR)-mediated immune response. The T cell hybridomas of the invention are typically constructed by fusing a naïve or early central memory T cell isolated from a mammalian subject with an immortalizing fusion partner (e.g. mammalian lymphoid tumor cell) to yield clonal T cell hybrids. The resulting T cell hybridomas exhibit antigen (Ag)-specific proliferation responsiveness over a background level of proliferation of the hybridomas. These hybridomas are useful for screening, identifying, and characterizing T cell immune modulatory agents, for example recombinant T cell receptor ligands (RTLs) and other agents that can modulate TCR-mediated T cell immune responses. Ag-specific proliferative response profiles exhibited by the T cell hybridomas of the invention show sufficient amplitude, sensitivity and fidelity to distinguish and/or quantify the presence and/or activity of a test RTL or other test modulatory agent in screening and sensitivity assays employing the hybridomas. These and other aspects of the invention yield powerful tools and methods for developing and characterizing novel RTLs and other immune modulatory agents for use in treating immune disorders, including a wide range of autoimmune diseases, in mammals.

The invention relates to the field of immunology. More particularly, the invention relates to hybridoma technology and related compositions and methods for developing and evaluating diagnostic and therapeutic tools for modifying immune responses in mammalian subjects, including for management of autoimmune diseases, such as multiple sclerosis.

### BACKGROUND OF THE INVENTION

Immune responses in mammals are mediated by a diverse collection of peripheral blood cells called leukocytes. These cells arise from hematopoietic stem cells, which undergo self-renewal and differentiation into two precursor lineages—the myeloid and lymphoid lines. Further differentiation occurs among these lineages to produce monocyte, eosinophil, neutrophil, basophil, megakaryocyte, and erythroid cells from the myeloid line, and T lymphocytes, B lymphocytes, and NK cells from the lymphoid line.

T lymphocyte differentiation occurs in the thymus and proceeds through prothymocyte, cortical thymocyte and medullary thymocyte intermediate stages, to produce various types of mature T cells. The principal T lymphocyte subtypes include CD8⁺ T cells (also known as cytotoxic/suppressor T cells or CTLs), which, when activated, have the capacity to lyse target cells, and CD4⁺ T cells (also known as T helper and T inducer cells), which, when activated, have the capacity to regulate other immune system cell types.

Immune system responses are elicited in several differing situations. The most frequent response is as a desirable protective response against infectious microorganisms. However, undesired immune responses can occur in the context of various autoimmune diseases, and following transplantation of foreign tissue. In the case autoimmune diseases, the body's own antigens present targets for autoreactive immune responses. Immune responses can also be initiated in vitro by mitogens, antibodies against certain receptors, and cognate antigens of T cells.

Immune responses are “transduced” from a stimulating event via a complex interaction of leukocyte cell types and regulatory molecules. The participating cell types and the nature of interactions between cell types may vary for different stimulating events. For example, immune responses against invading bacteria are often transduced by formation of complexes between an MHC Class II receptor and a bacterial antigen, which trigger activation of CD4⁺ T cells. By contrast, immune responses against viral infections are principally transduced by formation of MHC Class I/viral antigen complexes and subsequent activation of CD8⁺ cells.

Activation of T lymphocytes occurs naturally when the T cells interact with antigen-presenting cells (APCs) bearing cognate antigen (Ag) in the context of a major histocompatibility complex (MHC) protein. The specificity of T cell responses is conferred by a polymorphic, antigen-specific T cell receptor (TCR). In particular, most T cell activation involves recognition by an α/β heterodimeric TCR of antigen that has been processed and presented on the surface of APCs as peptides bound to MHC molecules. TCR α and β chains are each derived from multiple germline-encoded elements which undergo somatic recombination during T cell development. In the mouse, the repertoire of α chain germline genes is thought to number approximately 100 different variable (V α) segments, and also 100 different joining (J α) segments. TCR β chains are derived from a repertoire of gene segments estimated to include some 25 different Vβ elements, 12 Jβ genes, and two diversity (Dβ)elements. Random joining of these various intra α and β chain elements, along with combinatorial associations of different α and β chains, permit a considerable degree of TCR diversity. In addition, during the somatic process of V/J recombination (V/D/J for β chains), further diversity is created in the junctional regions through the addition of nongermline encoded nucleotides (N region). Given the number of different germline elements, the number of possible junctional region sequences, and the combinatory associations of the two chains, it has been estimated that the potential TCR repertoire is of the order of 1015-1022 specificities (Davis et al., Nature 334:395, 1988; Hunkapillar et al., Adv. Immunology 44:1, 1988)

In addition to the antigen specific TCR, a number of other cell surface proteins regulate T cell activation and impart sensitivity and flexibility to the immune response. These additional regulatory proteins include the surface antigens CD2, CD4 CD8 and lymphocyte function associated antigen. Such surface antigens are non-polymorphic molecules that increase the avidity with which a T cell interacts with APCs or target cells, and also play a role in signal transduction.

CD4 and CD8 molecules are expressed on mutually exclusive populations of mature T cells that bear TCRs specific for antigen in association with MHC class II, and MHC class I proteins, respectively. These molecules enhance the avidity with which a T cell binds antigen-bearing or target cells, and may also promote the interaction of the TCR with its appropriate antigen. Bierer et al., Ann. Rev. Immunol. 7:579-99, 1989.

MHC-restricted T lymphocyte interactions have been widely and extensively investigated. Cells of the T helper/inducer subset generally recognize antigen on the surface of APCs only in association with class II MHC gene products, which results in genetic restriction of antigen recognition. While the rules governing the activation of MHC-restricted T cells, and particularly of class II MHC-restricted T cells, have been well described, the underlying mechanisms are still being defined.

Despite the very large number of possible TCR specificities of T cells, a number of studies have shown that the major portion of the T cell response to numerous different protein antigens may be directed to a few “immunodominant” epitopes within the antigenic protein. In the context of autoimmune diseases, HLA-DR4-restricted T cell responses, and in some cases clinical signs of autoimmune disorder, have been demonstrated to be associated with specific proteins and/or immunodominant epitopes from these proteins, including, e.g., type II collagen (Rosloneic et al., J. Immunol. 160:2573-78, 1998; Andersson et al., Proc. Natl. Acad. Sci. USA 95:7574-79, 1998; and Fugger et al., Eur. J. Immunol. 26:928-33, 1996), and human cartilage Ag gp39 (Cope et al., Arthritis Rheum. 42:1497, 1999) associated with rheumatoid arthritis (RA), glutamic acid decarboxylase 65 (Patel et al., Proc. Natl. Acad. Sci. USA 94:8082-87, 1997; Wicker et al., J. Clin. Invest. 98:2597, 1996) and insulin (Congia et al., Proc. Natl. Acad. Sci. USA 95:3833-38, 1998) associated with Type 1 diabetes (insulin dependent diabetes mellitus or IDDM), and myelin oligodendrocyte glycoprotein (MOO) (Forsthuber et al., J. Immunol. 167:7119, 2001) associated with MS and an animal disease model for MS, experimental autoimmune encephalomyelitis (EAE). Similar findings have been reported for HLA-DR2-restricted T cell responses associated with myelin basic protein (MBP) (Madsen et al., Nat. Genet. 23:343, 1999), proteolipid protein (PLP) (Kawamura et al., J. Clin. Invest. 105:977, 2000), and MOG (Vandenbark et al., J. Immunol. 171:127-33, 2003).

In addition to the phenomenon of immunodominant peptide recognition, TCR utilization in autoimmune responses is often quite limited, despite a vast diversity of TCRs available in the T cell population. For example, limited TCR utilization has been reported in the immune response of Lewis rats immunized with myelin basic protein (MBP) to induce EAE. The T cell response thus induced is directed primarily to an immunodominant epitope contained within an encephalitogenic fragment of MBP comprising amino acids 66-88 of the protein (MBP 66-88). The T cell response is also highly restricted, dominated by T cells expressing TCR Vβ8 almost exclusively and Vα2 frequently (Burns et al., J. Exp. Med. 169:27, 1989). Similarly, Gold et al., J. Exp. Med. 174:1467-176, 1991 report conserved TCR Vα and Vβ utilization in EAE, wherein the TCR β chain sequences of T cell clones and hybridomas reactive to MBP 68-88 all utilized a Vβ8.2 segment, and exhibited other conserved structural features.

One very promising approach for regulating antigen-specific T cell responses in autoimmunity and in other contexts (e.g., graft rejection) is to reprogram or induce nonresponsiveness in T cells using recombinant or synthetic TCR ligands, or T cell modulatory drugs or other compounds that are agonists or antagonists for activation of TCRs by their cognate ligands. In this regard, various analogs of TCR ligands have been produced which comprise extracellular domains of class II MHC molecules linked to specific peptide targets. Several such constructs have been developed that involve natural or recombinant α1α2 and β1β2 MHC class II domains in association with various encephalitogenic or other pathogenic peptides covalently linked or noncovalently bound to the MHC II component to form a complex (Kozono et al., Nature 369:151, 1994; Fremont et al., Science 272:1001, 1996; Sharma et al., Proc. Natl. Acad. Sci. USA 88:11405, 1991; Nicolle et al., J. Clin. Invest. 93:1361, 1994; Spack et al., CNS Drug Rev. 4: 225, 1998). These molecular complexes bind not only to the TCR but also to the CD4 molecule on the T cell surface through the 132 MHC domain (Brogdon et al., J. Immunol. 161:5472, 1998), and have been reported to inhibit T cell activation and prevent EAE in rodents (Sharma et al., Proc. Natl. Acad. Sci. USA 88:11405, 1991; Spack et al., CNS Drub Rev. 4: 225, 1998; Steward et al., J. Allerg. Clin. Immun. 2:S117, 1997).

An even more promising design for TCR modulatory agents in this context are recombinant T cell receptor ligands (RTLs) that incorporate a minimal TCR interface, for example comprising only the α1 and β1 MHC domains (or otherwise excluding the β2 CD4-binding domain) covalently linked to peptide (Burrows et al., Prot Eng. 12:771, 1999). These RTL constructs have been shown to prevent and treat MBP-induced EAE in Lewis rats (Burrows et al., J. Immunol. 161:5987, 1998; Burrows et al., J. Immunol. 164:6366, 2000) and to inhibit activation and induce IL-10 secretion in human DR2-restricted T cell clones specific for MBP-85-95 or BCR-ABL b3a2 peptide (CABL) (Burrows et al., J. Immunol. 167:4386, 2001; Chang et al., J. Biol. Chem. 276:24170, 2001). Additional RTL constructs have been designed and tested by inventors in the instant application, which include a MOG-35-55/DR2 construct (VG312) shown to potently inhibit autoimmune responses and lead to immunological tolerance to the encephalitogenic MOG-35-55 peptide and reverse clinical and histological signs of EAE (Vandenbark et al., J. Immunol. 171:127-33, 2003). Numerous additional RTL constructs that are useful for modulating T cell immune responses and can be employed within the invention are described herein, below.

To evaluate the biological function and mechanisms of action of RTLs and other T cell modulatory agents, antigen-specific T cells bearing cognate TCRs have been used as target T cells for testing (see, e.g., Burrows et al., J. Immunol. 167:4386, 2001). However, a low frequency of Ag-specific T cells, varying levels of T cell Ag-specific responses, and a potential for uncontrolled interactions (e.g., with other, different cells) have significantly limited the scope of these investigations.

Two basic strategies have been devised to isolate and propagate lymphocyte lines and clones of defined specificity for evaluating T cell function and modulation. One approach has been to clonally expand and propagate normal immune lymphocytes through repetitive stimulation with antigen and/or growth factors. The second approach has been to produce immortalized lymphocyte hybrids by somatic cell hybridization with cancer cells (e.g., lymphoma or myeloma cells).

The latter approach of “hybridoma” technology was discovered by Kohler and Milstein (Nature 256:495, 1975), and was initially directed toward production of B lymphocyte hybrids (e.g., between B cells and immortal, plasmacytoma cells) to produce monoclonal antibodies. Subsequently, these methods were adapted toward production of functional, continuous lines of T lymphocytes.

Hybridomas are created from two cell populations by fusing the two cell types together with a fusogen, such as polyethylene glycol (PEG). The resulting fused cells (hybridomas) are isolated from unfused cells and used to establish continuous cell lines. Isolation of hybridomas typically involves the use of a selective medium, for example, hypoxanthine-aminopterin-thymidine (HAT) medium. The hybrid cells are then expanded and subcloned to generate a pure cell line, or monoculture.

Although the majority of hybridoma work to date has focused on B cells for producing monoclonal antibodies, the method has also been effectively used to make functional T cell hybrids. For example, Kappler, et al., (J. Exp. Med. 153:1198-1214, 1981) described murine T cell hybridomas made by fusing an interleukin-2 (IL-2)-producing, HAT-sensitive murine T cell tumor line with normal murine T cell blasts enriched in H-2 antigen-specific cells. The resulting hybrids could be induced to produce IL-2 by mitogen and/or antigens. Other early reports of T cell hybridoma production are provided by Grillot-Courvalin, et al. (Nature 292:844, 1981), and Greene, et al. (Clin. Res. 29:368A, 1981). Grillot-Courvalin, et al. made a T cell hybridoma by hybridizing normal peripheral blood T cells with a HAT-sensitive T cell lymphoma. Greene et al. made a suppressor T cell hybrid by fusing peripheral blood lymphocytes enriched for suppressor T cells (by treatment with the mitogen, concanavalin A (ConA) with a HAT-sensitive T leukemia cell line.

Hybridoma methodology has more recently been employed for production of antigen-specific, class II MHC-restricted, T-T hybridomas. Various class II MHC-restricted T-T hybridomas have been produced, and these hybrids have shown utility for various purposes, including the analysis of T cell receptor structure-function, cell-cell interactions, and T cell activation involving T helper cells (see, e.g., White et al., J. Immunol. 130:1033-37, 1983; Rock, K. L., “Functional T-cell Hybridoma”, in Hybridoma Technology in the Biosciences and Medicine, Ed. T. A. Springer, Plenum Press, N. Y., 1985; Allen et al., Proc. Natl. Acad. Sci. USA 81:2489-93, 1984; Allen et al., J. Exp. Med. 162:1264-68, 1985a; Allen et al., J. Immunol. 135:368-72, 1985b; Marrack et al., Adv. Immunol. 38:1, 1986; Jarboe et al., Infect. Immun. 52:326-30, 1986; Bierer et al, Ann. Rev. Immunol. 7: 579, 1989; Peccoud et al., EMBO J. 9:4215-23, 1990; Engleman et al., U.S. Pat. No. 4,950,598, issued Aug. 21, 1990; U.S. Pat. No. 5,019,384, issued May 28, 1991, and Sypek et al., Infect. Immun. 58:1146-52, 1990).

A survey of more recent literature on T cell hybridoma research includes the following reports:

White et al., (J. Immunol. 143:1822, 1989), and White et al. (J. Exp. Med. 177:119-25, 1993), generated hybridomas from a TCR-negative variant of the widely used AKR thymoma line BW5147. This BW5147 variant fusion partner does not express functional TCR alpha- and beta-chains (as determined by surface TCR staining). These cells were fused with normal T lymphocytes for study at the clonal level (without interference of functional, i.e., surface expressed, BW5147-derived TCR chains).

Gold et al., (J. Exp. Med. 174:1467-76, 1991) reported isolation of T cell hybridomas produced by fusion of lymphoblasts from MBP-specific T cell lines to a murine TCR α/β-(BW1100.129.237) cell line. The subject hybridomas exhibited IL-2 production following stimulation with MBP (whole protein or synthetic peptide MBP 68-88) in the presence of irradiated LEW spleen cells.

Michäelson et al., (Eur. J. Immunol. 22:1819-25, 1992) generated T hybridomas from rat CII-reactive T cell lines and myelin basic protein (MPB(89-101)-reactive T cells fused with the BW 5147 α-β-variant partner according to the method of White et al: (J Immunol. 143:1822, 1989). The reactivity of these T cell hybridomas was again assayed indirectly, using an IL-2-dependent CTLL cell line. This activity was inhibited by preincubation of spleen APCs with inhibitory peptides prior to stimulation of the hybridomas with stimulatory peptide.

Woods et al., (J. Exp. Med. 180:173-81, 1994) generated T cell hybridomas using T cells from a Tg murine subject in which the α1 and β1 domains of mouse I-E^{d} were replaced by the corresponding domains of human DRB1*0401 (Dr4Dw4) molecules (capable of presenting human class II-restricted peptides to mouse CD4-positive cells). The starting T cells were isolated from lymph nodes of the Tg mice after immunization with a synthetic peptide from influenza virus hemaglutinin, HA(307-319), and were fused with the BW 5147 α-β-variant partner. The hybridomas responded (as detected indirectly by IL-2 HT-2 bioassay) specifically to HA (107-319) presented by transgenic, but not nontransgenic, spleen cells, and exhibited DR4Dw4 restriction specificity.

Liu et al., (J. Exp. Med. 186:1441-50, 1997), and Crawford et al. (Immunity 8:675-82, 1998) generated T cell hybridomas specific for a well-studied combination of MHC+peptide (M^{k}+moth cytochrome c 88-103 (MCC)). Tg Mice were created that expressed genes coding for soluble IE^{k} covalently linked via a flexible linker at its NH2-tenninal end to peptides shown to bind intact IE^{k} protein (Kozono et al., Immunity 3:187-196, 1995). Tg mice injected with IE^{k} bound to an MCC peptide produced T cells that were fused with TCR α-/β-BW5147 variant thymoma cells. The resulting hybridomas expressed high levels of TCR and CD4, and were assayed for their ability to be bound by and react with react with IE^{k} and different concentrations of MCC in the presence of B10.BR spleen cells as APCs, or with a soluble, multimeric biotinylated IE^{k}MCC ligand complex. Hybridoma responses were again measured by indirect IL-2 assay, which reportedly showed that the hybridoma responses were CD4-dependent and IE^{k}-specific (as determined by anti-IE blockade).

Laufer et al., (J. Immunol. 162:5078-84, 1999) generated T hybridomas using splenocytes derived from K14 mice (a Tg murine model for inflammatory skin disease) stimulated with irradiated B 6 splenocytes and fused with the BW 5147 α-β-variant fusion partner. B6-reactive hybrids were selected as positive if they showed a response over background in indirect IL-2 activation assays using the IL-2-dependent indicator cell line HT-2. (See, also, Fan et al., Proc. Natl. Acad. Sci. USA 100:3386-91, 2003).

Fukui et al., (Proc. Natl. Acad. Sci. USA 97:13760-65, 2000) made T cell hybridomas specific for pigeon cytochrome c-derived peptide, 50V, restricted by I-A^{b}. The subject hybridomas were stimulated with irradiated LN cells and 50V peptide and assayed indirectly for IL-2 production using CTLL.

Chen et al., (J. Virol. 74:7587-99, 2000) made hepatitis B virus (HBV) e antigen (HBeAg)-specific T cell hybridomas from T cells generated in HBeAg Tg (B10 e/e) mice and also using the BW 5147 α-β-variant fusion partner. A panel of these hybridomas was used to identify an immunodominant (129-140/IA^{b}) epitope of HBeAg. TCR genes from selected hybridomas were cloned and sequenced to elucidate V gene usage, and further data showed that multiple TCRs recognized the 129-140/IA^{b} epitope. Some of these hybridomas were selected as candidate donors of TCR genes for generating TCR Tg mice.

Rosloneic et al., (J. Immunol. 160:2573-78, 1998) and Kotzin et al., (Proc. Nat. Acad. Sci. USA 97:291-96, 2000) also made T cell hybridomas using the BW5147 thymoma variant TCR α-/β-fusion partner. In the former study, the authors examined the function of a RA susceptibility allele HLA-DR4 (DRB1*0401) in presenting antigenic peptides derived from human type II collagen (hCII). hCII specific lymphocytes were obtained following immunization of mice transgenic (Tg) for a chimeric HLA/I-E DR4 (in which the HLA second domains are exchanged for I-E, enabling murine CD4 to interact with the chimeric molecule), and were fused with the variant BW5147 thymoma cells. The resulting hybridomas were screened for their ability to recognize hCII presented by DR4 and I-A^{f} presented by syngeneic spleen cells. This assay similarly involved indirect detection of hybridoma activation by IL-2 assay (measuring IL-2 titers indirectly through the proliferative response of co-cultured HT-2 cells).

In the Kotzin et al. study, hCII-specific T cell hybridomas were made by immunizing DR4 Tg mice (DRB1*0401) with human CII, and fusing T cells from the immunized Tg mice with the variant (TCR α-/β-) BW5147 thymoma cells. Two hybridomas (DR4hCII-38.8 and DR4hCII-11.5) specific for the dominant CII determinant (CII 263-270) were generated and assayed for their ability to bind a labeled, soluble, tetrameric CII peptide-DR4 complex. The CII DR4 tetramers bound in a specific manner to the T cell hybridomas that were previously shown (by indirect, IL-2 assay, as described by Rosloneic et al., supra) to respond to the major immunodominant determinant, CII 263-270.

In this same study, Kotzin et al. made additional T cell hybridomas transfected to express chimeric TCR genes of clonal CD4+ expansions identified in RA synovial cells. The chimeric TCR genes were introduced into TCR-negative and human CD4-expressing T cell hybridomas by electroporation, and the transfectants were screened for surface expression of TCR and shown to be functional by IL-2 release after stimulation with a Vβ-selective superantigen. These transfected hybridomas were control assayed for stimulation by CII(258-272) and HCgp39(263-275) peptides in the context of DR4 (*0401)-expressing APCs, and none reportedly responded.

Andersson et al., (Proc. Natl. Acad. Sci. USA 95:7574-79, 1998) used

HLA-DR (DRB1*0401, DRA1*0101), human CD4 Tg mice to generate T hybridomas responsive to an immunodominant T epitope in CII identified as CII 261-273, also using the variant TCR α-/β-BW5147 fusion partner. These hybridomas were incubated with a DRB1*0401-positive Epstein-Barr virus-transformed B cell line (Priess), as APCs and varying concentrations of the CII peptide, and activation was determined by IL-2 specific sandwich ELISA based on an Eu³⁺-labeled streptavidin detection system (Fugger et al., Eur. J. Immunol. 26:928-33, 1996).

Backlund et al., (Proc. Nat. Acad. Sci. USA 99:9960-65, 2002) used a humanized mouse model expressing HLA-DRB1*0401/DRA1*0101, human CD4, and human CII (huCII) on a background deficient of murine class II expression (Fugger et al., Proc. Natl. Acad. Sci. USA 91:6151 55, 1994; Malmström et al., Scand. J. Immunol. 45:670-77, 1997) to further elucidate the role and behavior of T cells in RA. From these studies the authors reported a dominant T cell response to glycosylated CII-glycopeptides in a cohort of severely affected RA-patients.

Maverakis et al., (Proc. Natl. Acad. Sci. USA 100:5342-47, 2003) made T hybridomas specific for an N-terminal autoantigenic peptide (Ac1-9) of myelin basic protein (MBP), again using the TCR α-/β-BW5147 variant fusion partner. Peptide-responsive hybridomas were also identified by indirect IL-2 production bioassay using HT-2 cells.

Strattman et al., (J. Clin. Invest. 112:902-14, 2003) reported production of additional T cell hybridomas using T cells from NOD mice (an art accepted model for insulin-dependent diabetes mellitus, or IDDM) (see also, Haskins et al., Diabetes 37:1444, 1988). These hybrids were generated by fusing BW5147 cells with T cells isolated from NOD mice immunized with a “2.5 mimotope” peptide with high agonistic activity for autoreactive BDC-2.5 T cells isolated from NOD mice and shown to be pathogenic in transfer experiments (Judkowski et al., J. Immunol. 166:908-17, 2001; Pankewycz et al., Eur. J. Immunol. 21:873-79, 1991; Daniel et al., Eur. J. Immunol. 25:1056-62, 1995); Haskins et al., Science 249:1433-36, 1990). These hybridomas were specifically bound by the 2.5 mimotope peptide complexed to A^{g7} to form an MHC-peptide tetramer complex (A^{g7} is the sole MHC class II molecule of NOD mice). As in previous studies, activation of these hybridomas in response to antigen (purified from pancreatic islets and presented by NOD splenocytes as APCs) was determined indirectly by IL-2 assays measuring proliferation of an IL-2 dependent cell line (NK) (Strattman et al., supra).

In a separate IDDM-related study by Patel et al., (Proc. Natl. Acad. Sci. USA 94:8082-87, 1997), the BW5147 α-/β-variant was used as a fusion partner with T cells isolated from DR0401, hCD4, I-AB (C-line) mice immunized with purified recombinant human glutamic acid decarboxylase 65 (GAD65) protein. Over 200 hybridomas thus produced were reported to be specific for GAD65 by using spleenic APCs from DR0401, hCD4, I-Aβ mice. The GAD-65-specific hybridomas were tested against pools of overlapping GAD65 peptides in the presence of APCs (Tg mouse spleen cells, or Epstein-Barr virus (EBV)-transformed lymphoblastoid cells), and hybridoma activation was assayed by an IL-2 sandwich immunoassay employing a streptavidin-europium detection system. This epitope mapping study revealed an immunodominant core motif of GAD65 (LYNIIKNREG), which included a DR0401 motif (YNIIKNREG) and a DR0405 motif (LYNIMNRE) (Rammensee et al., Immunogenetics 41:178-228, 1995). For each of 10 identified epitopes, the authors reported verified MHC restriction by antibody blocking experiments (anti-DR antibodies reportedly blocked hybridoma activation, whereas an isotype-matched control antibody did not affect the antigen-specific response).

In a related study, Congia et al., (Proc. Natl. Acad. Sci. USA 95:3833-38, 1998) fused T cells from DRB1*0401, hCD4+, I-Aβ^{0/0} mice immunized with purified recombinant human proinsulin or preproinsulin with TCR α-/β-variant BW5147 cells. A panel of hybridomas was initially screened using whole protein (insulin, proinsulin, or preproinsulin) and later using overlapping peptides, and hybridoma activation was indirectly measured by a europium-based IL-2 immunoassay. Using this panel of hybridomas, an immunodominant epitope (pgs. 73-90) was identified spanning the C peptide and the A chain, another epitope (pgs. 11-26) in the leader sequence, another spanning the leader and b chain (pgs. 20-36), and a fourth specific for the A chain (pgs. 85-101), indicating that the major T cell determinant of insulin and its prohormones is located in the precursor forms of the protein.

Yang et al., (Proc. Natl. Acad. Sci. USA 99:6204-6209, 2002) constructed an alternative “THZ” T cell hybridoma by fusing activated mouse CD4+ T cells with the BWZ cell line containing a reporter gene (lacZ) expressed under control of an element of the IL-2 promoter (nuclear factor of activated T cells (NFAT)). A single THZ clone was isolated that lacked TCR expression but maintained CD3 and CD4 expression. This hybridoma reportedly allows for detection of surface expression and specificity of TCRs on the hybridomas by assaying lacZ activation in response to antigen.

Recently, Wang et al. (J. Immunol. 171:1934-40, 2003) used a rat T cell hybridoma specific for guinea pig (GP)-MBP (originally developed by Gold et al., J. Exp. Med. 174:1467-76, 1991) as a clonal target to examine the function of RTLs. In this study, Wang and colleagues demonstrated that the T cell hybridoma was selectively, but only partially, activated to transduce early signaling through the TCR in an Ag-specific fashion.

The foregoing T cell hybridomas have essentially limitless growth potential and grow readily under standard culture conditions. Furthermore, the immunological activity of these hybridomas is not subject to cyclic fluctuations, as is seen with restimulated normal clones. T-inducer hybridomas produce lymphokines in response to T cell receptor (TCR) stimulation, which provides a useful assay for examining T cell activation events. Moreover, since T-inducer hybrids do not generally require costimulatory signals, it has been possible to stimulate them with fixed or disrupted antigen presenting cells as well as model membrane systems (see, e.g., Shimonkevitz, et al., J. Exp. Med. 158:303, 1983; Watts, et al., Proc. Natl. Acad. Sci. USA 81:1883, 1984). Therefore, antigen-specific class II MHC restricted T cell hybridomas have provided improved tools for analyzing the events in T cell Ag presentation and TCR-mediated regulation of T cell biology.

With respect to class I MHC-restricted hybridomas, these have also been made using immortalizing fusion partners. Whitaker et al., (J. Immunol. 129:900-03, for example, described the preparation of H-2 restricted, reovirus-specific cytotoxic T cell hybrids using BW5147 as a fusion partner. These hybrids, however, require a mitogenic lectin for stimulation and do not respond well to antigen and appropriate APCs alone. Other examples of class I MHC-restricted hybridomas generated with BW5147 cells have been reported by Kaufmann et al., (Proc. Natl. Acad. Sci. USA 78:2502, 1981), and by Endres et al., (J. Immunol. 131:1656, 1983). However, such hybrids are identified at low frequency and may represent “high affinity” T cells that are not CD8 dependent (see, e.g., MacDonald et al, Immunol. Rev. 68:89, 1982). Furthermore, the successful isolation of such hybridomas has been limited to those that are MHC reactive (e.g., allo- or auto-reactive) and has not been described for antigen-specific, MHC-restricted cells.

Despite the wide ranging and long enduring efforts evinced by the foregoing reports, the goals of developing useful, clonal T cell lines for analyzing T cell function and identifying and characterizing T cell modulatory agents, remain only partially fulfilled. The utility of existing T cell hybridomas for these purposes remains seriously limited. A principal drawback of current technologies in this regard relates to the inherently high proliferation potential of immortalizing T cell fusion partners for generating T hybridomas (as driven constitutively, for example, by oncogen promoters). Because of this high intrinsic proliferative capacity exhibited by the requisite fusion partner, assays to detect and characterize Ag-specific, TCR-mediated activation responses by T cell hybridomas have almost universally focused on a single indicator of T cell activation, IL-2 production. Regardless of the IL-2 assay format used, the resulting data are necessarily limited in terms of detecting, characterizing, and/or measuring meaningful T cell regulatory events. The same inherent limitation applies to other known T hybrid activation assays, for example as described by Yang et al., (Proc. Natl. Acad. Sci. USA 99:6204-09, 2002). In the Yang et al. study, “THZ” hybridomas have been characterized by a limited Ag-specific activation profile—characterized by lacZ reporter expression which is also linked to a discrete activation event of IL-2 gene activation.

A more comprehensive biological assay for T cell activation, indicative of a broader assemblage of T cell activation mechanisms and events, is therefore highly desired. A widely recognized T cell response in this context is represented by Ag-specific T cell proliferation, which response indicates a much broader and biologically significant activation profile than a single cytokine upregulation event. However, as noted above, previously developed T cell hybridomas have not been shown to exhibit useful, Ag-specific proliferative responses in this context, and available assays have focused almost exclusively on indirect measurements of IL-2-induced proliferation of cocultured cells. This deficiency may be attributed, at least in part, to the high background proliferation imparted to T cell hybridomas by the immortalizing fusion partner, which are required for generating clonal hybrids.

By virtue of their high constitutive or baseline proliferation capacity, existing T cell hybrids fail to exhibit meaningful, Ag-specific proliferation responses over background. More importantly, the masking baseline proliferative potential of known T cell hybridomas makes these clones refractory to studies of RTLs and other T cell modulatory agents. In particular, these hybrids are not amenable to assays for detecting and/or measuring agonistic or antagonistic effects of RTLs and other TCR modulatory agents (i.e., in comparison to a “control” proliferative response elicited and observed in the hybrids following cognate Ag-stimulation in the absence of the RTL or other modulatory agent). Even if such a control or baseline proliferative response above background could be discerned in a known T cell hybridoma after Ag-stimulation, the subject hybridoma would not exhibit the necessary fidelity and sensitivity of an Ag-specific proliferative response to permit detection and/or quantification of a more attenuated, anti-proliferative or pro-proliferative effect (e.g., as would have to be discerned following pre- or post-incubation of Ag-stimulated hybrids with a test RTL or other T cell modulatory agent, in comparison to such control or baseline proliferative response in the absence of the test agent).

Accordingly, there remains a compelling need in the art for T cell hybridomas that exhibit a readily-detectable and quantifiable, Ag-specific proliferation response over a background level of proliferation of the hybridomas (inclusive of a resting or constitutive proliferation rate, and of a non-specific activation proliferation level after stimulation by mitogen and/or APCs in the absence of cognate antigen).

A related need exists for T cell hybridomas that exhibit an Ag-specific proliferation response profile suitable for detecting and/or measuring agonistic and antagonistic effects of RTLs and other TCR modulatory agents on Ag-induced proliferation of the hybridomas.

It is therefore an object of the invention to provide T cell hybridomas that exhibit a biologically meaningful Ag-specific proliferation response over a background level of proliferation of the hybridomas. It is a further object of the invention to provide T cell hybridomas that exhibit an Ag-specific proliferative response following cognate Ag-stimulation in the absence of a test RTL or other test modulatory agent, that is of sufficient sensitivity and fidelity to distinguish and/or quantify the presence and/or activity of the test RTL or other test modulatory agent in a screening or sensitivity assay culture of the hybridomas.

Yet another object of the present invention is to provide quantitative and sensitive assays for the identification and characterization of T cell modulatory RTLs and other T cell modulatory agents useful for modulating TCR-mediated, T cell immune responses in investigative, diagnostic, and/or therapeutic applications.

The present invention fulfills these objects and satisfies additional objects and advantages, as will become apparent from the following description.

### SUMMARY OF EXEMPLARY EMBODIMENTS

The present invention provides T cell hybrids and related assay systems that allow for the screening, design, construction, and characterization of novel immune modulatory agents, including recombinant T cell ligands (RTLs).

The T cell hybrids of the invention are typically produced by fusing a mammalian T cell that expresses a T cell receptor (TCR) with a mammalian fusion partner cell to yield a clonal hybrid. The T cell hybrids exhibit an antigen (Ag)-specific, TCR-mediated proliferative response when stimulated by cognate Ag. This Ag-specific proliferative response can be detected above a background or resting proliferation rate of the T cell hybrids.

The T cell hybrids of the invention also display an Ag-specific, TCR-mediated proliferative response following contact with cognate, which can be detectably inhibited or stimulated by contacting the T cell hybrid with a TCR antagonist or TCR agonist. The Ag-specific, TCR-mediated proliferation kinetics of the T cell hybrids permit use of the T cell hybrids in screening and quantitative assays to detect the presence, concentration and/or activity of TCR antagonists and agonists.

The invention thus provides a sensitive biological assay for TCR-mediated T cell activation, allowing for reliable detection of both Ag-stimulated and agonist- or antagonist-mediated effects on TCR function.

The methods and compositions of the invention provide proliferation-based assays that indicate a broader, more biologically significant range of TCR-activation events. These methods and compositions are therefore readily incorporated in assays to screen, identify, and characterize immune modulatory agents that can alter TCR-mediated T cell immune responses. Within these novel assay tools and methods, the T cell hybrids of the invention exhibit modified, Ag-specific, TCR-mediated proliferation responses that accurately and reproducibly indicate a presence, quantity, and/or activity level of a selected RTL or other TCR modulatory agents in contact with the T cell hybrid. In related aspects of the invention, the T cell hybrids are employed in assays for detecting and/or measuring the presence, concentration and/or activity of a T cell agonist or T cell antagonist that may be added as a test agent to a culture of the T cell hybrids and observed for their modulatory effects on TCR-mediated activation of hybrid proliferative and other activation responses. In other aspects of the invention, the T cell hybrids are employed in a range of investigative, diagnostic, and clinical applications.

### BRIEF DESCRIPTION OF THE DRAWINGS

FIG. 1. T cell lines specific for mouse (m)MOG-35-55 and human (h)MOG-35-55 peptides from splenocytes of HLA-DRB1*1501-Tg mice. A) Splenocytes from mMOG-35-55 peptide-immunized DRB1*1501-Tg mice at 2^{nd} stimulation with 0.08 to 50 μg/ml mMOG-35-55 peptide; B) Splenocytes from hMOG-35-55 peptide-immunized 1501-Tg mice at 2^{nd} stimulation with 0.08 to 50 μg/ml hMOG-35-55 peptide; C) A T cell line selected from a DRB1*1501-Tg mouse immunized with mMOG-35-55 peptide at 2^{nd} stimulation reactive to mMOG-35-55 and cross-reactive with hMOG-35-55 peptides in a reciprocal dose-dependent fashion.

FIG. 2. Expression of TCR V gene segments in BW5147, BW5147 variant and T cell hybrids. TCR BV and AV gene expression was detected by RT-PCR. Top panel: BW5147.G.1.4 thymoma cell line purchased from ATCC (#TIB 48). Second panel: H6-1 hyrid clone, a T cell from mMOG-35-55 peptide-immunized DRB1*1501-Tg mice fused with a BW5147 cell. This hybrid was specific for mMOG-35-55 peptide (SI=4×) and cross-reactive with hMOG-35-55 peptide (SI=4×). Third panel: BW5147 variant cell line. Fourth panel: H2-1 hybrid clone, a T cell from hMOG-35-55 peptide-immunized DRB1*1501-Tg mice fused with a BW5147 variant cell. This hybrid was specific for hMOG-35-55 peptide (SI=12×) and cross-reactive with mMOG-35-55 peptide (SI=10×). Nomenclature: A10=AV19S1; BM.A=AV18S2; BM.B=AV17S3; BW.B=AV16S1P.

FIG. 3. Surface and intracellular staining of anti-TCR beta and anti-AV11 on BW5147, BW5147 variant and T cell hybrids. A) 0.5×10⁶ cells of BW5147, BW5147 variant, H6-1 and H2-1 were stained with anti-TCR beta-Cy-Chrome or anti-mouse IgG 2a-Cy-Chrome. B) 1×10⁶ cells from each of the above samples were incubated with Brefeldin A for 6 hr followed by surface staining with anti-CD4-FITC, fixation, and permeabilization. Permeabilized cells were further stained with anti-TCR beta-Cy-Chrome. C) 1×10⁶ cells from H2-1, H6-1 and BW5147 were stained with anti-AV11-FITC for 45 min followed by FACScan analysis.

FIG. 4. Phenotyping of MOG-35-55 peptide-specific T cell hybrids. A) 1×10⁶ cells of H2-1, H6-1 and BW5147 were stained with anti-CD3-FITC alone, or anti-CD4-PE plus anti-CD8-FITC, or anti-mouse IgG1-PE. B) Intracellular expression of IL-2, TNF-α and IFN-γ by ConA-activated T cell hybrid H2-1.

FIG. 5. Ag-specific responses by T cell hybrids. A) H2-1 selected from 384 isolates of H2 hybrid was tested for its response to hMOG-35-55 peptide in both a direct proliferation in the presence of DRB1*1501-transfected L cells and an indirect assay using 50% supernatant on CTLL-2 cells for 24 hr. B) H6 and A5 hybrids were derived from mMOG-35-55 peptide-specific T cells and tested for responses to mMOG-35-55 peptide and ConA in an indirect assay. C) H2 hybrid was tested for cross-reactivity to mMOG-35-55 peptide and both H6 and A5 hybrids were tested for cross-reactivity to hMOG-35-55 peptide in the presence of DRB1*1501-transfected A PC. 50% supernatants were assayed. D) H2 hybrid was cultured with syngeneic APC in the presence of 10 μg/ml hMOG-35-55, MBP-85-99, or PLP-95-116 peptides for 72 hr and 50% supernatants were assayed. Student's t test was used. E) H2 hybrid cells (40,000 per well in triplicate) were added with either 1×10⁵ irradiated syngeneic APC or 20,000 irradiated (4500 rad) transfected L cells expressing DRB1 *1501 or DRB5*0101 in the presence of 10 μg/ml either MOG-35-55 peptides or 5 μg/ml ConA for 72 hr, and the supernatants were assayed on CTLL-2 cells for 24 hr.

FIG. 6. Biological functions of hMOG-35-55-specific RTL detected by H2-1 hybrid in vitro and verified in hMOG-35-55-induced EAE. A) 2×10⁵/well H2-1 hybrid cells in triplicate were cultured in 2 mM Tris-containing medium alone, or with 8 uM RTL VG1000 or 8 uM RTL340 (specific for MBP-85-99) for 24, 48, 72 and 96 hr. 0.5 uCi [³H]Thymidine was incorporated for the last 6 hr of each culturing time. Proliferation of cultured H2-1 hybrid cells was compared among three different culture conditions at each culturing time. Asterisks indicate significantly reduced responses (p<0.05, Student's t test) between RTL-treated vs. buffer treated hybrid cells. B) Aliquotted H2-1 hybrid cells or Al hybrid cells were collected after 24 and 96 hr co-culture with 8 uM RTL VG1000 and washed three x with RPMI and re-plated in a new culture plate with 1×10⁵ hybrid cells per well, plus irradiated DRB1*1501-transfectant APC with and without 10 ug/ml hMOG-35-55 peptide (for H2-1), or irradiated rat thymocytes with and without 10 ug/ml GP-BP-69-89 peptide (for A1). After 48 hr culture, 100 ul supernatant was collected from each well and transferred correspondingly into a new plate in which 5000 CTLL-2 cells suspended in 100 ul per well were already placed. 0.5 uCi [³H]Thymidine was incorporated for the last 6 hr of a 24 hr culture. Proliferation of CTLL cells was measured by net cpm over background cpm that was less than 1000-1500. C) Nine DRB1*1502-Tg male and female mice were immunized s.c. with hMOG-35-55 peptide in CFA. In addition, mice were given pertussis toxin (Ptx) on days 0 and 2 postimmunization. After onset of clinical signs, three mice were treated i.v. with 100 ul of 1 mg/ml (40 uM) RTL VG1000 daily for 8 days and the other 6 mice were injected i.v. with 100 ul of 10 mM Tris buffer daily for 8 days. The average daily EAE score was determined for each group of mice by summing the individual scores and dividing by the number of mice in the group.

### DESCRIPTION OF EXEMPLARY MODES OF INVENTION

The present invention provides novel mammalian T cell hybrids that are useful in a variety of methods and compositions. In one embodiment of the invention, the T cell hybrids described herein are used in screening and other assay compositions and methods to screen, identify, develop and/or characterize immune modulatory agents that regulate T cell function. In related embodiments, the T cell hybrids are employed in assays for detecting and/or measuring the presence, concentration and/or activity of T cell agonists or T cell antagonists that modulate T cell receptor (TCR)-mediated, T cell immune responses. Within exemplary embodiments, the T cell hybrids are used for identifying and/or characterizing recombinant T cell receptor ligands (RTLs) that regulate T cell function through the TCR. In other aspects of the invention, the T cell hybrids are employed in a range of investigative, diagnostic, and clinical applications.

The T cell hybrids of the invention are typically produced by fusing a mammalian T cell that expresses a TCR with a mammalian fusion partner cell to yield a clonal T cell hybrid. Surprisingly, the clonal T cell hybrids of the invention exhibit an antigen (Ag)-specific, TCR-mediated proliferative response when the hybrids are stimulated by cognate Ag. This proliferative response may occur upon stimulation by Ag alone, and/or following stimulation by Ag in the presence of a suitable, typically allogeneic, Ag-presenting cell (APC). This Ag-specific proliferative response can be detected above a background or resting proliferation rate of the T cell hybrids. For example, the T cell hybrids of the invention will exhibit a detectable increase in proliferation in response to stimulation by cognate Ag and/or APC, which can be accurately distinguished from a proliferation rate of the T hybrids without Ag stimulation, and/or following non-specific stimulation (e.g., by a mitogen, such as concanavalin A (ConA), or by a cytokine, such as IL-2).

In more specific embodiments, the T cell hybrids of the invention display an Ag-specific, TCR-mediated proliferative response following contact with their cognate Ag (Ag alone, or Ag combined with APC), which can be detectably inhibited or stimulated by contacting the T cell hybrid with a TCR antagonist or TCR agonist. According to this aspect of the invention, the Ag-specific, TCR-mediated proliferation kinetics of the T cell hybrids exhibit sufficient fidelity and reproducibility to permit use of the T cell hybrids in screening and quantitative assays to detect the presence, concentration and/or activity of TCR antagonists and agonists. When the T cell hybrids are contacted with a selected TCR agonist or antagonist, optionally before or after Ag-stimulation, a modified, Ag-specific, TCR-mediated proliferation response of the hybrid cells can be observed. Detection or measurement of this modified proliferation response indicates a presence, quantity, and/or activity level of the T cell antagonist or T cell agonist in contact with the T cell hybrid. In exemplary embodiments, the T cell hybrids exhibit an Ag-specific, TCR-mediated proliferative response following contact with the cognate Ag and APC that can be detectably inhibited or stimulated by contacting the T cell hybrid with a selected RTL. This aspect of the invention yields sensitive, quantitative methods and compositions useful for designing, screening, selecting, developing, and characterizing novel or modified RTLs.

The invention thus provides a sensitive and comprehensive biological assay for TCR-mediated T cell activation. Because the T cell hybrids exhibit such unexpected amplitude and fidelity of proliferative responsiveness relative to background, they allow for reliable detection of both Ag-stimulated and agonist- or antagonist-mediated effects on TCR function. This permits broad-mechanistic identification and characterization of TCR-mediated T cell activation. The related T cell/TCR activation assays of the invention are therefore significantly more meaningful and useful than assays based on previously-described T cell hybrids—which have not been shown to exhibit useful, Ag-specific proliferative responses or agonist/antagonist effects on Ag-stimulated proliferation (due, at least in part, to the masking influences of their high constitutive or baseline proliferation capacity). Whereas prior T cell hybrid-based assays focused necessarily on limited, often indirect activation indices (e.g., indirect measurement of interleukin (IL)-2 production), the methods and compositions of the invention provide proliferation-based assays that indicate a broader, more biologically significant range of TCR-activation events. The methods and compositions of the invention are therefore uniquely amenable for screening, identifying, and characterizing RTLs and other TCR modulatory agents useful for modulating T cell activity in vivo, for example to prevent or treat autoimmune diseases. Within these novel assay tools and methods, the T cell hybrids of the invention exhibit modified, Ag-specific, TCR-mediated proliferation responses that accurately and reproducibly indicate a presence, quantity, and/or activity level of a selected RTL or other TCR modulatory agents in contact with the T cell hybrid.

The T cell hybrids of the invention typically display a proliferation response to their cognate Ag in the presence of APCs that is two-fold or greater than a control or baseline level of proliferation of the hybrids. This proliferation response may be measured in comparison to a baseline proliferation level of the T cell hybrids under similar culture conditions but in the absence of cognate Ag and APCs. Alternatively, the Ag-specific proliferation response may be exhibited over a non-specific control activation level induced, for example, by cytokines (e.g., IL-2), APCs alone, APCs in combination with a different, non-specific antigen, non-specific antigen alone, and/or mitogen (e.g., ConA). In certain embodiments, the T cell hybrids of the invention exhibit a 3-fold or greater proliferative response to cognate Ag/APC stimulation above baseline or above proliferation levels of non-specific activation controls. In other embodiments, the Ag/APC proliferative response is 5-fold or greater than baseline or non-specifically activated T hybrid proliferation levels. In certain embodiments the Ag/APC proliferative response is 8-fold or greater, 10-fold or greater, and up to 15-fold or greater compared to baseline or non-specifically activated T hybrid proliferation levels. These amplitudes, and the fidelity and reproducibility, of the Ag-specific proliferation profiles of the T hybrids of the invention provide for effective employment of the T hybrids in sensitive, quantitative assays and related compositions useful for designing, screening, selecting, developing, and characterizing novel or modified RTLs and other TCR modulatory agents.

For use within the methods and compositions of the invention, antigen presenting cells (APCs) include dendritic cells, macrophages, B cells, and other cells that can process and present antigenic peptides in association with class I or class II MHC molecules and deliver a co-stimulatory signal necessary for T cell activation.

The T cell hybrids of the invention typically express CD3 and CD4, and often display a Thl cytokine expression profile (see, e.g., Kerlero, J. Autoimmunity 11:287-99, 1998). In certain embodiments, the T cell hybrids express detectable levels of IL-2, tumor necrosis factor (TNF)-α, and interferon (IFN)-γ, either constitutively or in response to non-specific (e.g., ConA) stimulation and/or Ag-specific stimulation. Commonly, the T cell hybrids exhibit significantly enhanced (e.g., 20-30%, 30-45%, or greater increased levels) of IL-2, TNF-α, TNF-β, and/or IFN-γ expression in response to non-specific stimulation and/or Ag-specific stimulation. In more detailed embodiments, the T hybrid cells may express low or undetectable levels of IFN-γ, consitutively, and the levels of IFN-γ expression may be lower constitutively or following stimulation than levels ekhibited by T effector memory (Tem) cells (see below). In related embodiments, the T hybrid cells express low or undetectable levels of Th2 cytokines, including IL-4, IL-5 and/or IL-10. Other Th2 cytokines not typically expressed at substantial levels (i.e., at comparable levels to other known Th2 cells) by T cell hybrids of the invention include, but are not limited to, IL-6 and IL-13. Frequently, Th2 cytokine expression is not significantly elevated, and is more commonly low or undetectable, following non-specific stimulation (e.g., following exposure of the cells to ConA).

In additional embodiments of the invention, the T cell that is used to form the T cell hybrid is selected from naïve T cells or central memory T cells (Tcms). Typically, the starting T cells are obtained from a bone marrow, spleen, or draining lymph node of a mammalian subject following immunization of the subject with the cognate Ag.

Identification and characterization of naïve and Tcm starting T cells follows generally the teachings of Gudmundsdottir et al., J. Immunol. 162:5212-23, 1999; Sallusto et al., Nature 401:708-712, 1999; Gramaglia et al., J. Immunol. 162:1333-1338, 1999; Campbell et al., J. Immunol. 166:877-84, 2001, Younes et al., J. Exp. Med. 198:1909-22, 2003, Geginat et al., Pathol. Biol. 51:64-66, 2003, and Morel et al., Eur. J. Immunol. 33:3212-19, 2003. The use of nave and/or Tcm starting T cells strongly contributes to the amplitude and fidelity of the Ag-specific proliferation response profile of the resulting T hybrid cells.

Certain starting T cells for use in constructing the T cell hybrids of the invention are selected for positive expression of CD45. Typically, CD45 expression is high in naïve T cells, and low or undetectable in Tcm and T effector memory (Tem) cells. Within these embodiments, the starting T cell may be a non-human mammalian T cell that is CD45RB+. Alternatively, the starting T cell may be a human T cell that is CD45RA+. In either case, a CD45+ phenotype will often be marked by CD45 expression at levels equal to, or slightly to moderately diminished from, levels of CD45 expression characteristic of naïve T cells (i.e., obtained from the same subject or species), and substantially higher than CD45 expression levels found in Tcm or Tem T cells. CD45+ T cells for use within the invention include activated naïve T cells which may be harvested from primary lymphoid organs of animals immunized with cognate Ag. Activated naïve T cells most commonly used as starting T cells express a CD45+ phenotype as described above, but may otherwise depart phenotypically from other nave T cells in expression of one or more other markers indicative of naïve T cell development, activation, and/or differentiation.

Within other aspects of the invention, the starting T cells are selected for positive expression of CD62L. Typically, CD62L expression is high in naïve T cells, and low or undetectable in Tem cells. Within these embodiments, CD62L expression will be often be exhibited at levels equal to, or slightly (e.g., 10-20%) to moderately (20-45%) diminished from, levels of CD62L expression characteristic of naïve T cells and/or Tcm cells, and substantially greater than (e.g., increased by 50% or more compared to) CD62L expression levels found in Tem cells. CD62L+ T cells for use within the invention include activated naïve T cells which may be harvested from primary lymphoid organs of animals immunized with cognate Ag. CD62L+ T cells most commonly used as starting T cells express a CD62L+ phenotype as described above, but may otherwise depart phenotypically from naïve T cells and or Tcm cells in expression of one or more other markers indicative of naïve T cell and/or Tem cell development, activation, and/or differentiation.

Within other aspects of the invention, the starting T cells are selected for positive expression of CD27. Typically, CD27 expression is high in naïve T cells and Tem cells, and low or undetectable in Tem cells. Within these embodiments, a CD27+ phenotype is marked by CD27 expression at levels equal to, or slightly (e.g., 10-20%) to moderately (20-45%) diminished from, levels of CD27 expression characteristic of naïve T cells and/or Tem cells, and substantially greater than (e.g., increased by 50% or more compared to) CD27 expression levels found in Tem cells. CD27+ T cells for use within the invention include activated naïve T cells and Tem cells which may be harvested from primary lymphoid organs of animals immunized with cognate Ag. Activated naïve T cells and Tem cells most commonly used as starting T cells express a CD27+phenotype as described above, but may otherwise depart phenotypically from naïve T cells and/or Tem cells in expression of one or more other markers indicative of naïve T cell and/or Tem cell development, activation, and/or differentiation.

Within other aspects of the invention, the starting T cells are selected for positive expression of CCR7. Typically, CCR7 expression is high in naïve T cells and Tem cells, and low or undetectable in Tem cells. Within these embodiments, a CCR7+ phenotype is marked by CCR7 expression at levels equal to, or slightly (e.g., 10-20%) to moderately (20-45%) diminished from, levels of CCR7 expression characteristic of naïve T cells and/or Tem cells, and substantially greater than (e.g., increased by 50% or more compared to) CCR7 expression levels found in Tem cells. CCR7+ T cells for use within the invention include activated naïve T cells and Tcm cells which may be harvested from primary lymphoid organs of animals immunized with cognate Ag. Activated naïve T cells and Tcm cells most commonly used as starting T cells express a CCR7+ phenotype as described above, but may otherwise depart phenotypically from naïve T cells and/or Tcm cells in expression of one or more other markers indicative of naïve T cell and/or Tcm cell development, activation, and/or differentiation.

Within other aspects of the invention, the starting T cells are selected for positive expression of lymphotoxin αβ (LT αβ). Typically, LT αβ expression is detectable 24-72 hours after activation of naïve T cells, and declines thereafter to become relatively low in Tem cells (see, e.g., Gudmundsdottir et al., J. Immunol. 162:5212-23, 1999). Within these embodiments, a LT αβ+ phenotype is marked by LT αβ expression at levels equal to, or slightly (e.g., 10-20%) to moderately (20-45%) diminished from, levels of LT αβ expression characteristic of activated naïve T cells and/or Tem cells, and substantially greater than (e.g., increased by 50% or more compared to) LT αβ expression levels found in Tem cells. LT αβ+ T cells for use within the invention include activated naïve T cells and Tem cells which may be harvested from primary lymphoid organs of animals immunized with cognate Ag. Activated naïve T cells and Tem cells most commonly used as starting T cells express a LT αβ+ phenotype as described above, but may otherwise depart phenotypically from naïve T cells and/or Tem cells in expression of one or more other markers indicative of naïve T cell and/or Tem cell development, activation, and/or differentiation.

Within other aspects of the invention, the starting T cells are selected for negative or low expression of the T cell differentiation marker CD44. Typically, CD44 expression is low or undetectable in naïve T cells, and comparably high in Tem cells. Within these embodiments, a CD44-phenotype will be marked by CD44 expression at levels equal to, or slightly (e.g., 10-20%) to moderately (20-45%) increased from, levels of CD44 expression characteristic of naïve T cells, and substantially lower than (e.g., less than 50% compared to) CD44 expression levels found in Tem cells. These CD44− T cells for use within the invention may be harvested from primary lymphoid organs of animals immunized with cognate Ag. Activated naïve, CD44− T cells most commonly used as starting T cells express a CD44− phenotype as described above, but may otherwise depart phenotypically from naïve T cells and/or Tcm cells in expression of one or more other markers indicative of naïve T cell and/or Tcm cell development, activation, and/or differentiation.

Within other aspects of the invention, the starting T cells are selected for negative or low expression of the T cell differentiation marker CD49d. Typically, CD49d expression is low or undetectable in naïve T cells, and comparably high in Tem cells. Within these embodiments, a CD49d− phenotype will be marked by CD49d expression at levels equal to, or slightly (e.g., 10-20%) to moderately (20-45%) increased from, levels of CD49d expression characteristic of naïve T cells and/or Tcm cells, and substantially lower than (e.g., less than 50% compared to) CD49d expression levels found in Tem cells. These CD49d− T cells for use within the invention may be harvested from primary lymphoid organs of animals immunized with cognate Ag. CD49d− T cells most commonly used as starting T cells express a CD49d− phenotype as described above, but may otherwise depart phenotypically from activated naïve T cells and/or Tcm cells in expression of one or more other markers indicative of naïve T cell and/or Tcm cell development, activation, and/or differentiation.

Within other aspects of the invention, the starting T cells are selected for negative or low expression of the T cell differentiation marker CD40L. Typically, CD40L expression is low or undetectable in unactivated, naïve T cells, and is rapidly upregulated upon T cell activation to become comparably high in Tem cells. Within these embodiments, a CD40L− phenotype will be marked by CD40L expression at levels equal to, or slightly (e.g., 10-20%) to moderately (20-45%) increased from, levels of CD40L expression characteristic of naïve T cells and/or Tcm cells, and substantially lower than (e.g., less than 50% compared to) CD40L expression levels found in Tem cells. These CD40L− T cells for use within the invention may be harvested from primary lymphoid organs of animals immunized with cognate Ag. CD40L− T cells most commonly used as starting T cells express a CD40L− phenotype as described above, but may otherwise depart phenotypically from naïve T cells and/or Tcm cells in expression of one or more other markers indicative of naïve T cell and/or Tem cell development, activation, and/or differentiation.

Within other aspects of the invention, the starting T cells are selected for negative or low expression of the T cell differentiation marker LIGHT, a recently identified member of the TNF superfamily (see, e.g., Morel et al., Eur. J. Immunol. 33:3212-19, 2003). Typically, LIGHT expression is low or undetectable in unactivated, naïve T cells, and is rapidly upregulated upon T cell activation to become comparably high in Tem cells. Within these embodiments, a LIGHT- phenotype will be marked by LIGHT expression at levels equal to, or slightly (e.g., 10-20%) to moderately (20-45%) increased from, levels of LIGHT expression characteristic of naïve T cells and/or Tcm cells, and substantially lower than (e.g., less than 50% compared to) LIGHT expression levels found in Tem cells. These LIGHT− T cells for use within the invention may be harvested from primary lymphoid organs of animals immunized with cognate Ag. LIGHT− T cells most commonly used as starting T cells express a LIGHT− phenotype as described above, but may otherwise depart phenotypically from naïve T cells and/or Tem cells in expression of one or more other markers indicative of naïve T cell and/or Tem cell development, activation, and/or differentiation.

Within other aspects of the invention, the starting T cells are phenotypically characterized by exhibiting a negative or low proliferative response to IL-7 and or IL-15 cytokines. Typically, naïve T cells and Tem cells exhibit a negative or low proliferative response to IL-7 and or IL-15, whereas Tem cells exhibit a highly efficient proliferative response to IL-7 and IL-15 cytokines (see, e.g., Geginat et al., Pathologie Biologie 51:64-66, 2003. Within these embodiments, the starting T cells will exhibit a proliferative response to IL-7 and IL-15 that is equal to, or slightly (e.g., 10-20%) to moderately (20-45%) increased from, levels of proliferation exhibited by naïve T cells and/or Tem cells in response to exposure to IL-7 and IL-15, and substantially lower than (e.g., less than 50% compared to) IL-7- and/or IL-15-induced proliferation levels exhibited by Tem cells. These IL-7- and/or IL-15-unresponsive T cells for use within the invention may be harvested from primary lymphoid organs of animals immunized with cognate Ag. IL-7- and/or IL-15-unresponsive T cells most commonly used as starting T cells exhibit a IL-7- and/or IL-15-unresponsive phenotype as described above, but may otherwise depart phenotypically from naïve T cells and/or Tem cells in expression of one or more other markers or proliferative response indices indicative of naïve T cell and/or Tcm cell development, activation, and/or differentiation.

Within additional aspects of the invention, the starting T cell used for production of the T cell hybrid is an Ag-specific T cell which has been Ag-stimulated in vitro by contacting the T cell with the cognate Ag. The starting T cell may be initially stimulated with Ag in vitro, or restimulated after collection of the T cell from a mammalian host immunized with the cognate Ag. Restimulation in this context is not required, but one or more rounds of Ag stimulation after collection of T cells from an immunized subject may be undertaken prior to fusion of the T cell with the mammalian fusion partner cell. Antigen stimulation in vitro may be achieved with or without concurrent contact of the starting T cell with an APC.

The T lymphocytes which can be used for fusion with the mammalian cell fusion partner are not otherwise particularly limited. Examples of useful T cells include those obtained from the bone marrow, lymph nodes, spleen, thoracic ducts, tonsils, peripheral blood, and thymus. Most often the T cells will be obtained from the bone marrow, lymph nodes, or spleen, although for convenience and reduction of morbidity in human donors, peripheral blood mononuclear leukocytes (PBLs) may be used. If T cells that produce specific immunoregulatory agents or mediators of cellular immunity are desired, normal T lymphocytes may be cultured in a medium containing an inducer that induces production of such agents or mediators prior to or after fusion. The concentration of inducer in the medium will depend upon the particular inducer and the cell concentration.

T lymphocytes can be isolated and or purified by various separating methods which are known, such as conventional physical methods, chemical methods and the adherence method to surface membranes, and can be used for fusion in accordance with the method described herein.

Ag-specific T lymphocytes are most often obtained by immunizing a mammalian host with the cognate Ag. The cognate Ag may be a naturally-occurring or synthetic peptide Ag (e.g., comprising a T cell epitope), a portion of a protein containing a peptide Ag, an entire protein containing one or more Ags, or a cell, cellular component, (e.g., membrane preparation), substrate, vehicle, or carrier comprising the Ag. The Ag may comprise one or more T cell epitope(s), and may include epitopes from different sources (e.g., a fusion protein or conjugate of multiple epitopes from the same, or different, protein, cell, or organism).

For immunization, the cognate Ag is administered to the mammalian subject to elicit an Ag-specific immune response in the subject. Immunization can be achieved, for example, by intravenous or subcutaneous injection of the Ag, optionally conjugated with a haptenating compound or hapten, or coupled to a carrier, membrane, or cell surface, or by live or attenuated infection or inoculation of cells (e.g., viral or bacterial pathogens, cancer cells, etc.) comprising the subject Ag in an immunogenic state. After a period following immunization sufficient for T cell activation, the lymphocytes may be isolated from the immunized host and enriched for antigen-specific T lymphocytes prior to fusion, according to various well known methods.

Within alternate embodiments of the invention, a naïve population of T cells can be removed and primed for Ag-specific response in vitro. Several strategies known in the art have been employed to enrich antigen-specific T lymphocytes. One strategy is to supply the requisite signals for activation and/or clonal expansion in vitro. Under these conditions, Ag-specific cells increase in number and irrelevant cells are diluted or die out. After a single restimulation with antigen, frequencies of greater than 1:100 can be achieved. With preferential fusion of activated T cells, specific hybrids can be as frequent as one in five. Starting T lymphocytes may optionally be exposed to antigen/APC stimulation, and/or helper factors prior to fusion. The latter will be generated in cultures from T inducer cell stimulation. If these cells have been depleted, they can be reconstituted or a source of helper factor added. Supernatant from mitogen-stimulated T cells (from which mitogen has been removed or inactivated) or from secondary mixed lymphocyte cultures are convenient sources of helper factors.

The T cells to be used in the fusion can also be alloreactive T lymphocytes, activated, for example, in vitro by mixed lymphocyte reaction (MLR). MLR involves a reaction in which lymphocytes from a first (responder) strain are mixed with lymphocytes from a second (stimulator) strain. The stimulator strain bears allogeneic MHC molecules, and is typically irradiated or treated with mitomycin C to render them incapable of dividing. After a suitable period of incubation, the responder cells are tested for Ag-specific response activity against cells of the stimulator strain bearing allogeneic MHC molecules.

Useful antigens for generation of Ag-specific starting T cells include, but are not limited to, peptides comprising an immunodominant epitope associated with a mammalian immune disorder. Among the contemplated immune disorders in this context are autoimmune diseases, inflammatory disorders, allergic conditions, cutaneous immune disorders, transplant rejection conditions, and graft versus host disease (GVHD). Other cognate Ags of interest include tumor antigens, and antigens of pathogenic agents (e.g., viral and bacterial pathogens).

Within more detailed embodiments of the invention, Ag-specific T lymphocytes are generated against cognate Ags comprising immunodominant epitopes associated with an autoimmune disease. For example, Ag-specific T lymphocytes may be generated against a variety of reported, MHC class II-restricted, immunodominant Ags associated with multiple sclerosis (MS), rheumatoid arthritis (RA), insulin-dependent diabetes mellitus (IDDM), chronic beryllium disease, autoimmune uveitis, sarcoidosis, systemic lupus erythromatosis, myasthenia gravis, Pemphigus vulgaris, Sjogren's syndrome, Addison's disease, autoimmune hepatitis, Graves disease, inflammatory bowel disease/Crohn's disease, and celiac disease. Alternatively, Ag-specific T lymphocytes may be generated against a variety of reported, MHC class I-restricted, immunodominant Ags associated with psoriasis, ankylosing spondylitis, Reiter's disease, and uveitis.

Exemplary aspects of the invention are directed toward the autoimmune disease MS. Within exemplary embodiments, the cognate Ag comprises an immunodominant, MS-associated epitope selected from a protein, peptide or epitope(s) of MOG (see, e.g., Forsthuber et al., J. Immunol. 167:7119, 2001; Vandenbark et al., J. Immunol. 171:127-33, 2003), MBP (see, e.g., Madsen et al., Nat. Genet. 23:343, 1999), and or PLP (see, e.g., Kawamura et al., J. Clin. Invest. 105:977, 2000). Other prospective Ags that may be important in MS and therefore useful within the invention are described for myelin-associated glycoprotein (MAG), non-myelin nervous system antigens, including the S10013 protein, and glial fibrillary acidic protein (GFAP), and non-neural specific antigens, such as heat shock proteins (hsps, including the small hsp αB crystalline), transaldolase, and 2′,3′-cyclic nucleotide 3′-phosphodiesterase (CNPase) (see, e.g., Kerlero J. Autoimmunity 11:287-99, 1998).

Exemplary cognate Ags comprising immunodominant T cell epitopes associated with MS for use within the invention include, but are not limited to huMOG-1-22, huMOG-35-55, huMOG-huMOG-1-22, huMOG-34-54, huMOG-63-87, huMOG-64-96, huMOG-92-106, murine (mu)-MOG-1-30, muMOG-35-55, muMOG-81-110, muMOG-91-110, rat (rt)-MOG-1-20, rtMOG-35-55, rtMOG-74-90, hu-MBP-85-99, hu-MBP-86-99, hu-MBP-87-99, guinea pig (Gp)-MBP-72-89, rt-MBP-72-89, and PLP-139-151.

Other known, cognate Ags for use in generating Ag-specific T cells, T cell hybrids, and RTLs within the invention include, for example, proteins, peptides and specific immunodominant epitopes mapped and reported for type II collagen (Rosloneic et al., J. Immunol. 160:2573-78, 1998; Andersson et al., Proc. Natl. Acad. Sci. USA 95:7574-79, 1998; and Fugger et al., Eur. J. Immunol. 26:928-33, 1996), human cartilage Ag gp39 (Cope et al., Arthritis Rheum. 42:1497, 1999), and E. coli heat shock peptide that are associated with RA, and glutamic acid decarboxylase 65 (Patel et al., Proc. Natl. Acad. Sci. USA 94:8082-87, 1997; Wicker et al., J. Clin. Invest. 98:2597, 1996), insulin (Congia et al., Proc. Natl. Acad. Sci. USA 95:3833-38, 1998), and insulinoma antigen that are associated with IDDM. Yet additional cognate Ags for use in generating Ag-specific T cells, T cell hybrids, and RTLs within the invention include, for example, proteins, peptides and specific immunodominant epitopes mapped and reported for Addison's disease (adrenocortical 21-hydroxylase), autoimmune hepatitis (anti-smc antibodies (Abs), anti-mitochondrial Abs, and anti-liver/anti-kidney Abs), celiac disease (gluten peptides), Graves disease (TSH receptor), myasthenia gravis (acetylcholine receptor), pemphigus vulgaris (epidermal cadherin, keratinocyte cell surface antigens), and systemic lupus erythematosus (nucleoproteins).

In addition to the phenomenon of immunodominant peptide recognition, TCR utilization in autoimmune responses is often quite limited, despite a vast diversity of TCRs available in the T cell population. For example, limited TCR utilization has been reported in the immune response of Lewis rats immunized with myelin basic protein (MBP) to induce EAE. The T cell response thus induced is directed primarily to an immunodominant epitope contained within an encephalitogenic fragment of MBP comprising amino acids 66-88 of the protein (MBP 66-88). The T cell response is also highly restricted, dominated by T cells expressing TCR Vβ8 almost exclusively and Vα2 frequently (Burns et al., J. Exp. Med. 169:27, 1989). Similarly, Gold et al., J. Exp. Med. 174:1467-76, 1991 report conserved TCR Vα and Vβ utilization in EAE, wherein the TCR β chain sequences of T cell clones and hybrids reactive to MBP 68-88 all utilized a Vβ8.2 segment, and exhibited other conserved structural features.

In accordance with the limited TCR repertoire involved in autoimmunity, and in relation to other desired attributes and features of the invention, the T cell hybrids described herein will typically possess defined TCR structural and functional features. In certain embodiments, the starting T cell will express one or more TCR gene(s) encoded by a transgene (i.e., including genes introduced into animal germline cells by transgenesis, and recombinantly engineered and expressed genes introduced into cells by recombinant expression vectors yielding transformation of the recipient cells to express the transgenic or recombinant TCR). In related aspects, one or more recombinant TCR gene(s) may be introduced into the starting T cell or hybrid by transduction or transfection using a recombinant expression vector that encodes the TCR gene(s) of interest. Typically, the subject TCR gene will be a TCR that specifically binds a cognate Ag (for example an Ag associated with a T cell autoimmune response or activity) and/or mediates T cell activation in an Ag-specific manner.

For methods and composition directed toward TCR structure-function analysis and rational design (i.e., including TCR design, engineering, construction, screening and characterization) the T cell or T cell hybrid may be express all or part of a TCR α chain, a TCRβ chain, a TCRγ chain, and/or a TCRΔ chain. Often, the TCR transgene or recombinant vector will be introduced into a RAG-deficient or RAG-null Tg mammalian subject or transformed recipient cell. For example, the TCR-encoding vector may be transduced or transfected into a mammalian cell that has a mutation in a RAG-1 and/or RAG-2 gene that renders the cell null or defective for RAG-1 and/or RAG-2 expression (see, e.g., Katz, et al., Cell 74:1089 1100, 1993; Gonzalez et al., Immunity 7:873 883, 1997).

In more detailed embodiments, the one or more TCR gene(s) that may be introduced transgenically or recombinantly into a subject or target cell for TCR expression will encode a selected TCR segment, chain, or complete receptor. TCR components of interest for expression in this regard include TCR components identified, for example, as mediating Ag-specific responses of T cells directed toward an Ag of interest, for example an immunodominant epitope associated with an autoimmune disease. Exemplary TCRs include TCRs, TCR segments, and TCR domains encoded by a selected AV or BV gene. For example, transgenic or recombinant TCRs of interest may include a TCR, segment, or domain encoded by a BV8, Vα2 AV11, or AV17 gene. Any AV or BV gene/gene product can be adapted in this context for use within the method and compositions of the invention.

In various exemplary embodiments, TCRs may be cloned from autoreactive T cells or T hybrids and further engineered for use within the foregoing methods and compositions. Often, the TCR will be engineered to incorporate modifications for structure-function analyses, for example the TCR may be modified at a putative Ag- or MHC-interactive domain or binding interface to incorporate site-directed mutations to develop and map novel receptor target sequences (e.g., for development of TCR agonists and antagonists). Various methods for TCR identification, cloning, mutation, recombinant expression, structure-function mapping, and screening (e.g., to screen small molecule drugs that modulate TCR binding or activation) are well known and widely practiced in the art (see, e.g., Pankewycz et al., Eur. J. Immunol. 21:873 879, 1991; Katz et al., Cell 74:1089-1100, 1993; Daniel et al., Eur. J. Immunol. 25:1056 1062, 1995; Haskins et al., Diabetes 37:1444 1448, 1988; Haskins et al., Proc. Natl. Acad. Sci. USA 86:8000 8004, 1989; Haskins et al., Science 249:1433 1436, 1990; and Nakano et al., J. Exp. Med. 173:1091-1097, 1991; and Gonzalez et al., Immunity 7, 873 883, 1997).

Exemplifying these complementary methods and tools for use within the invention, Strattman et al., (J. Clin. Invest. 112:902-14, 2003) describe isolation and transgenic expression of TCRs from an autoreactive, BDC-2.5 T cell line derived from a diabetic female NOD mouse. This cell line is a CD4+ Th1 clone that displays a Vβ4/Vα1 (AV1S5) TCR heterodimer (Candeias et al., Proc. Natl. Acad. Sci. USA 88:6167-6170, 1991) and was shown to greatly accelerate disease when transferred to young animals. Mice transgenic for the BDC-2.5 TCR were produced and employed by Strattman et al. for various purposes in their study. The cDNA for the α and β chains of the BDC-2.5 TCR was obtained by RT-PCR according to known methods. PCR products were subcloned into a suitable cloning vector, sequenced, and subcloned into a metallothionein promoter-based fly expression vector. Each of the final constructs coded for the α1α2 and the β1β2 domains, respectively, followed by a linker sequence (SSADL), a thrombin site

(LVPRGS), a leucine zipper (acidic for the a chain, basic for the β chain), and a hexahistidine tag. Vectors were transfected into SC2 cells, and stable cell lines and clones were established. Soluble TCRs were purified from culture supernatants according to known methods. In addition, Strattman et al. describe useful methods for cell preparation, cell staining, flow cytometry analysis, and adoptive transfer. In one modified assay, Strattman et al. constructed an MHC-mimetic peptide with high agonistic activity for BDC-2.5 T cells, which was complexed to the Ag7 molecule in an MHC multimer molecule (Crawford et al., Immunity 8:675 682, 1998; Altman et al., Science 274:94 96, 1996) and assayed against. Tetramers of the Ag7/mimotope complex were shown to be specific for BDC-2.5 T cells.

In the same context, the invention provides for expression of transgenic and recombinant TCRs by animals (e.g., animals from which starting T cells are obtained for fusion), by T cells, and by T cell hybrids that may be assayed (e.g., for TCR binding and/or activation) against TCR ligands, including native and/or recombinant ligands (e.g., RTLs, see below), and these materials and methods can be used in screens to identify other compounds (e.g., peptides and small molecule drugs) that affect Ag-specific TCR binding and/or activation) Further details regarding the methods and tools that can be employed in this aspect of the invention are provided elsewhere herein and/or among the references cited herein, which are all incorporated herein by reference). According to one exemplary procedure, Strattman and colleagues produced a functional transgenic BDC-2.5 TCR mice and transformed transgenic BDC-2.5 TCR T cell hybrids, as well as an Ag7/2.5 mimotope complex. This synthetic ligand for the BDC-2.5 T cells was isolated from a chemically synthesized random-peptide library based on its ability to stimulate the BDC-2.5 T cells (as measured by IL2 assay). Alternatively, the TCR transfectants were screened for surface expression of TCR and verified to be functional as determined by release of IL-2 after stimulation with an appropriate VP-selective superantigen (presented by autologous Epstein-Barr virus-transformed B cells). In a related report, Fan et al., Proc Natl Acad Sci USA 100: 3386-91, 2003, constructed TCR Tg mice that provide a model of inflammatory skin disease, in which keratinocytes activate and are the primary target of autoreactive CD4+ T cells. In particular, the investigators generated keratin 14 (K14)-Aβb mice expressing MHC class II only on thymic cortical epithelium, and showed that the CD4+ T cells from these K14-Aβb mice fail to undergo negative selection and thus have significant autoreactivity. The TCR genes from an autoreactive K14-Aβb CD4 T cell hybridoma were cloned to produce the TCR Tg mouse, and the Tg cells were negatively selected in WT C57BL/6 mice but not in 2-2-3/K14-Aβb mice. Additional methods and tools for use within these aspects of the present invention are described, for example by Laufer et al. (Nature 383:81-85, 1996), Martin et al. (Cell 84:543-50, 1996), Laufer et al. (J. Immunol. 162:5078-84, 1999), (Berg et al., Mol. Cell. Biol. 8:5459-69, 1988), Ho et al., (J. Exp. Med. 179:1539-49, 1994), Rojo et al., (J. Immunol. 140:1081-88, 1988), Kersh et al. (J. Immunol. 161:585-93, 1998), Haskins et al. (Diabetes 37:1444-48, 1988), Bensinger et al. (J. Exp. Med. 194:427-38, 2001), Wells et al. (J. Clin. Invest. 100:3173-83, 1997), and Vassar et al. (Genes Dev. 5:714-27, 1991).

[1 06] In accordance with other aspects of the invention, the T cell hybrids described herein will typically possess defined MHC structural and functional features. In certain embodiments, the starting T cell will express one or more native or recombinant (including wild type or selected mutant) MHC gene(s) encoded by a transgene. In related aspects, one or more recombinant MHC gene(s) may be introduced into the starting T cell or hybrid by transduction or transfection using a recombinant expression vector that encodes the MHC gene(s) of interest. Typically, the subject MHC gene will be a MHC II gene that encodes a portion of an MHC II complex that specifically binds a cognate Ag (for example an Ag associated with a T cell autoimmune response or activity) and/or mediates T cell activation in an Ag-specific manner.

For methods and composition directed toward MHC structure-function analysis and rational design the T cell or T cell hybrid may express all or part of a MHC I or MHC II molecule, for example a MHC II α chain, or MHC II β chain. Alternatively, the T cell or T cell hybrid may express a recombinant MHC molecule, including recombinant MHC molecules comprising one or more selected portions or domains of an MHC chain. In exemplary embodiments, the subject recombinant MHC molecule may comprise a single chain MHC II construct including selected, peptide binding and/or TCR-interactive portions of the corresponding native MHC II molecule (e.g., as described herein for single chain MHC II molecules associated or bound with cognate Ag peptides, comprising an RTL).

Within more detailed embodiments of the invention, the starting T cell is transgenic for a selected MHC genotype. Typically, these starting T cells are obtained from a MHC Tg subject. Exemplary MHC Tg subjects include a large assemblage of Tg murine subjects known and available in the art that are Tg for a selected MHC class II (MHC II) genotype. Commonly, the selected MHC II genotype will be a mutant MHC II isotype associated with an immune disorder, frequently an autoimmune disease. The MHC II genotype will typically specify an MHC II that functions to restrict Ag-specific, TCR-mediated immune responses. Exemplary T cell hybrids of the invention will therefore often be constructed using a starting T cell that is transgenic for a mutant HLA-DR, HLA-DP, or HLA-DQ isotype that contributes to a mammalian immune disorder, for example an autoimmune disease, inflammatory disorder, allergic condition, cutaneous immune disorder (psoriasis, atopic dermatitis, cutaneous T cell lymphoma), transplant rejection condition, or graft versus host disease (GVHD). Numerous such isotypes are known and characterized in the art in association with autoimmune disorders, including MS, RA, IDDM, chronic beryllium disease, autoimmune uveitis, sarcoidosis, systemic lupus erythromatosis, myasthenia gravis, and celiac disease.

In related embodiments, the mutant HLA-DR, HLA-DP, or HLA-DQ isotype is selected from a mutant HLA-DR1, HLA-DR2, HLA-DR3, HLA-DR4, HLA-DP2, HLA-DQ6, or HLA-DQ8 isotype. Exemplary mutant MHC II isotypes in this context include, but are not limited to, DRB1*0101; DRB1*0301, DRB1*0401, DRB1*0405, DRB1*1501, DRB4*0101, DRB5*0101, DPA1*0101, DPA1*0103, DPB1*0201, DPB1*0401, DQA1*0102, DQA1*0501, DQB1*0201, DQB1*0301, DQB1*0302, DQB1*0602, and DQB1*0604.

Among the features of these novel T cell hybrid expressing a MHC II transgene (and/or a selected mutant MHC isotype), the Ag-specific, TCR-mediated proliferative response of these hybrid will typically be restricted by the corresponding HLA-DR, HLA-DP, or HLA-DQ MHC II.

For constructing T cell hybrids of the invention, a variety of useful fusion partner cells are provided. Typically, the mammalian fusion partner cell is from a tumor or other cancer cell line. Most commonly the fusion partner cell is a clonal myeloid or T cell line. In alternate embodiments, the mammalian fusion partner cell or the T cell hybrid is transformed into a clonal cell line by transduction or transfection of an immortalizing gene into said cell. Commonly used immortalizing genes in this context include various oncogenes, such as ras. In related embodiments, the mammalian fusion partner cell or the T cell hybrid is transformed into a clonal cell line by viral transformation, for example by Epstein Barr virus (EBV) transformation (see, e.g., U.S. Pat. No. 4,720,459, issued Jan. 19, 1988).

The starting T cells and the mammalian fusion partner cells for generating the T cell hybrids may be from any mammalian species and may be from the same species or two different species in a given fusion. Commonly, both the starting T cell and mammalian fusion partner cell will be of the same species, for example both human or both murine cells. Typically the fusion partner cell will be deficient or null for TCR expression, for example they will not exhibit surface expression of intact, functional TCRs, or they will be genotypically null for one or more TCR genes.

In more detailed embodiments, the starting T cells are obtained from murine, rat, rabbit, or other suitable non-human, mammalian subject. Commonly, the fusion partner will correspond to the starting T cell in species and/or individual origin. In exemplary embodiments, the T cell hybrid is formed by fusion of a murine T cell, typically from a Tg murine background, with a murine T cell line. Various murine T cell cancer lines (e.g., lymphoma and thymoma lines) are useful within this context. In exemplary embodiments, a variant of the well known murine thymoma line BW5147 is employed as the fusion partner. For T cell fusions, the BW5147 thymoma is the most widely used tumor partner cell line (Chien et al., Nature 312:31-35, 1984; Yague et al., Cell 42:81-87, 1985; Blackman et al., Immunol. Rev. 101:5-19, 1986; Lee et al., J. Immunol. 140:1665-75, 1988). Although this tumor cell line does not bear surface TCR, existing endogenous TCR genes can be expressed as surface receptors after fusion with a normal T cell (Yague et al., Cell 42:81-87, 1985; Blackman et al., Cell 47:349-57, 1986). White et al. isolated two variants of the BW5147 cells that can no longer express functional TCR α.- and β-chains (White et al., J. Immunol. 143:1822-25, 1989). These variants have been used to determine the structure of TCR δ chain (Born et al., Nature 330:572-74, 1987), and to provide an appropriate tool for analysis of the TCR repertoire used in response to any given Ag, including self-determinants (Blackman et al., Immunol. Rev. 101:5-19, 1988; Kappler et al., Nature 332:35-40, 1988). Suitable BW5147 variants for use within the invention will often be a TCR-deficient or null BW5147 variant. For example, a desired BW5147 variant may be negative for cell surface expression of TCRα and TCRβ.

To further facilitate production and use of the T hybrids of the invention, mammalian fusion partner cells will most commonly be selected that are deficient or null for CD3 and/or CD4 expression. In certain embodiments, the fusion partner will not detectably express one or both of the CD3 and/or CD4 products.

In other detailed embodiments, the starting T cells are human T cells, and the mammalian fusion partner cell is a human T leukemia line. The T leukemia cell line is commonly a AH-sensitive mutant derived from the Jurkat human T leukemia cell line. The derivation of the Jurkat line was originally achieved by successively culturing parent Jurkat cells in media containing graded concentrations of 6-thioguanine. Prior to culturing, the parent cells may be optionally exposed to a mutagenic agent, e.g., an alkylating agent such as ethylmethane-sulfonate, to increase the frequency of mutants. Surviving cells exhibit an inability to incorporate 6-thioguanine suggesting they are deficient in hypoxanthine-guanine phosphoribosyl transferase (HGPRT).

An exemplary Jurkat T leukemia line for use within the invention is the J3R7 T leukemia cell line deposited at the American Type Culture Collection, (ATCC), Manasass, Va. on 2 Sep., 1982, which has been assigned the ATCC number CRL 8169. Other examples of well known, publicly available human cell lines for use as immortalizing fusion partners with T cells include human KE37.3.2, KE 37, MOLT3, MOLT 4, CCRF-CEM, and HuT 78 cells (see, e.g., U.S. Pat. No. 4,665,032, issued May 12, 1987).

To construct a parent leukemia cell line, the cells may be cultivated in successive incubations using, for example, graded concentrations of 6-thioguanine. The initial 6-thioguanine concentration is preferably about 10⁻⁵ M and is progressively increased to about 10⁻⁴ M. The total incubation time will typically be about 6 to about 12 weeks with the medium changed weekly. Accordingly, the number of incubations will usually be about 6 to 12. All incubations are carried out at approximately physiological temperature (e.g., about 37° C.). Conventional nutrient media such as RPMI 1640 may be used in these cultivations supplemented with 10% fetal calf serum (FCS). Each incubation will usually take about one week, with viable cells being isolated before the succeeding incubation. Sufficient cells are used in each incubation to ensure the likelihood of obtaining viable cells at the end of the incubation period. The number of cells per flask (25 cc volume) will usually be about 10⁷. The fastest growing 6-thioguanine resistant clones are chosen for use in the fusions. Such clones will typically have a doubling time in the range of about 18 to 24 hr. If necessary, the clones may be expanded prior to fusion to provide a suitable supply of cells for the fusion.

Fusion of the starting T cells and mammalian cell fusion partners can be carried out according to various well-known methods. Typically, the T cell and the mammalian fusion partner cell are fused to form the T cell hybrid by contacting the T cell and the fusion partner cell in the presence of a fusogen. In exemplary embodiments, the fusogen is a polyethylene glycol (PEG), for example PEG 1500 (Roch Diagnostics, Mannheim, Germany). Alternatively, the fusogen may be a virus, which can be any of a number of well known fusogenic viral strains, for example Sendai virus.

To accomplish the T cell fusions, a ratio of starting T cells to fusion partner cells can vary according to standard protocols. After fusion, unfused fusion partner cells die off after a few days in culture, and the fused cells can be separated from the unfused starting T cells by various known methods. In one exemplary method, the post-fusion cell mixture containing the fused as well as the parental cells may be incubated in a medium containing the selective reagent for a period of time sufficient to eliminate most of the unfused cells. For instance, a number of tumor cell lines are sensitive to HAT due to lack of functional hypoxanthine-guanine phosphoribosyl transferase (“HGPRT”). Fused cells formed by starting T cells and these tumor cell lines become resistant to HAT, as the starting T cells contribute functional HGPRT. Thus, a HAT selection can be performed after fusion to eliminate unfused parental cells.

In the event that the fused T hybrid cells lose certain starting T cell characteristics, such as expression of a selected TCR, they (i.e., primary fused cells) can be refused with the same or different starting T cells to restore a desired phenotype. Such refused cells (i.e., secondary fused cells) may be particularly potent or sensitive within the methods and compositions of the invention.

Although HAT fusion is a useful fusion and selection within the invention, alternative processes may be employed that avoid potential growth inhibitory effects of thymidine in HAT medium. Fox, et al., (Cancer Res 40:1718-21, 1980) report that the inhibitory effect of the thymidine can possibly be alleviated by the addition of deoxycytidine. Alternatives to HAT, such as a hypoxanthine-azaserine-thymidine medium have been used in B cell fusions to increase the yield of hybrids. More recently, Engleman et al. (U.S. Pat. No. 4,950,598, issued Aug. 21, 1990) reported fusing a HAT-sensitive human T leukemia line with a normal human T lymphocyte to produce human T cell hybridomas that were stable and produced IL-2 constitutively. In addition Engleman et al., reported that T cell fusions could be improved by fusing an azaserine-hypoxanthine sensitive human T leukemia (Jurkat) cell line with normal human T cells and culturing the hybrids in a selective azaserine-hypoxanthine medium—avoiding potential toxic effects on growth attributed to thymidine-containing selective media. Cultures of subclones from these fusions also reportedly exhibited stable, continuous growth and continuous production of IL-2.

For use within the invention, AH-sensitive T leukemia cells and starting (normal) T lymphocytes are fused by combining the two in a medium containing a fusogen. Polyethylene glycol (PEG) of about 1500 daltons is a preferred fusogen, but other PEGs of molecular weight in the range of about 1000 to 4000 daltons or other fusogens may be used. The fusogen will normally be present at between about 40% and 60% (v/v), preferably at about 50% (v/v), in the medium. The medium is preferably free of serum. The starting T cells and T leukemia cells will usually be combined at ratios of 2:1 to 1:10 with the total cells each being in the range of about 10⁶ to 5×10⁷. A volume of 0.5 to 1 ml of fusion medium per 10⁸ cells is often used. The cells are normally exposed to the fusion medium for about 5 to 10 minutes, often for about 7 to 8 minutes. A preferred fusion technique involves suspending the cells in the fusion medium followed by centrifugation of the cells while in contact with fusion medium. The fusion may be carried out at room temperature, typically with the fusogen prewarmed to 37° C.

Following the fusion the cells are separated from the fusion medium and washed repeatedly with fusogen-free medium to remove residual fusogen. The cells are then resuspended in the selective AH medium. The AH medium consists of a standard cell culture medium, such as RPMI or Dulbecco's medium, containing about 1 to about 20, preferably about 10, .μg azaserine per ml. The concentration of hypoxanthine in the medium is about 100 μM. The medium will also preferably contain serum, typically FCS, at a level of about 10% to 15% (v/v), an appropriate antibiotic such as penicillin/streptomycin and L-glutamine. Azaserine is a diazo analog of L-glutamine. Its main effect is an irreversible binding to various L-glutamine amino-transferases which are necessary in de novo purine biosynthesis. Its effect on pyrimidine synthesis is minimal and reversible with the addition of ademine. The use of azaserine in the selective medium avoids the necessity of using aminopterin which is an anti-folic acid antagonist and inhibits both purine and pyrimidine synthesis. Therefore, the use of azaserine eliminates the need for thymidine as an exogenous source of pyrimidine in the medium. This results in increased fusion efficiency and an increased rate of hybrid growth.

The success of T cell fusions and the stability of the resulting hybrids may be verified by a variety of known methods, for example including chromosomal analysis of the hybrids by conventional fixation and staining techniques. The resulting stable hybrid cells may be grown in vitro or in vivo by various known techniques. Production of immunoregulatory agents or mediators may be enhanced in the hybrids by adding inducers to the culture medium. In vivo cell growth may be effected by introducing the hybrid clones into a suitable immunodeficient host, such as nude mice or heavily irradiated mice. The clones will grow as ascites-producing tumors in such hosts. Compounds produced by the hybrids may be isolated from the culture medium, ascites fluid, or serum, as the case may be, by known separation and purification techniques such as ammonium sulfate precipitation, dialysis, chromatography, and gel electrophoresis.

The T cell hybrids of the present invention are useful in the analysis of the properties of individual T cells, as well as the underlying cellular and molecular events important in their activation. For example, the hybrids are well adapted for analyzing individual T cell specificities. In addition, the hybrids can be used to identify and characterize molecular events and constituents involved in T cell activation, including TCR binding and activation, and TCR-mediated T cell activation responses. For example, class II restricted T cell hybrids of the invention are useful for identifying, mapping, and characterizing determinants on cognate Ags, and on synthetic analogs and derivatives of cognate Ags, including peptide components of RTLs. Likewise, class II restricted T cell hybrids of the invention are useful for identifying, mapping, and characterizing determinants binding and activation sites or domains on TCRs that are critical to Ag-specific T cell activation. The hybrids are also useful for determining the roles of MHC molecules, and for mapping the structural and functional domains (e.g., peptide and TCR interactive sites) on MHC molecules and MHC components of RTLs, involved in antigen processing, antigen presentation, and/or MHC-TCR interactions.

In addition, the present invention provides for a broad range of manipulations of the hybrid cells, for example culturing, Ag-stimulation, and exposure to test immune modulatory agents under defined conditions, transformation of the hybrid cells (or starting T cells or fusion partner cells), for example, to express immune modulatory molecules, costimulation regulatory molecules, neuroprotective and/or neuroregenerative factors, growth factors, transgenic or recombinantly-modified TCRs, and exposure of the hybrid cells to prospective immune modulatory agents, for example small molecule drugs and RTLs (e.g., having novel or modified peptide and/or MHC components) to screen and characterize TCR-modulatory and/or T cell modulatory agents.

One very promising approach for regulating antigen-specific T cell responses in autoimmunity and in other contexts (e.g., graft rejection) is to reprogram or induce nonresponsiveness in T cells using recombinant or synthetic TCR ligands, or T cell modulatory drugs or other compounds that are agonists or antagonists for activation of TCRs by their cognate ligands. In this regard, various analogs of TCR ligands have been produced which comprise extracellular domains of class II MHC molecules linked to specific peptide targets. Several such constructs have been developed that involve natural or recombinant α1α2 and β1β2 MHC class II domains in association with various encephalitogenic or other pathogenic peptides covalently linked or noncovalently bound to the MHC II component to form a complex (Kozono et al., Nature 369:151, 1994; Fremont et al., Science 272:1001, 1996; Sharma et al., Proc. Natl. Acad. Sci. USA 88:11405, 1991; Nicolle et al., J. Clin. Invest. 93:1361, 1994; Spack et al., CNS Drug Rev. 4:225, 1998). These molecular complexes bind not only to the TCR but also to the CD4 molecule on the T cell surface through the β2 MHC domain (Brogdon et al., J. Immunol. 161:5472, 1998), and have been reported to inhibit T cell activation and prevent EAE in rodents (Sharma et al., Proc. Natl. Acad. Sci. USA 88:11405, 1991; Spack et al., CNS Drub Rev. 4:225, 1998; Steward et al., J. Allerg. Clin. Immun. 2:S117, 1997).

An even more promising design for TCR modulatory agents in this context are recombinant T cell receptor ligands (RTLs) that incorporate a minimal TCR interface, for example comprising only the al and 131 MHC domains (or otherwise excluding the β2 CD4-binding domain) covalently linked to peptide (Burrows et al., Prot Eng. 12:771, 1999). These RTL constructs have been shown to prevent and treat MBP-induced EAE in Lewis rats (Burrows et al., J. Immunol. 161:5987, 1998; Burrows et al., J. Immunol. 164:6366, 2000) and to inhibit activation and induce IL-10 secretion in human DR2-restricted T cell clones specific for MBP-85-95 or BCR-ABL b3a2 peptide (CABL) (Burrows et al., J. Immunol. 167:4386, 2001; Chang et al., J. Biol. Chem. 276:24170, 2001). Additional RTL constructs have been designed and tested by inventors in the instant application, which include a MOG-35-55/DR2 construct (VG312) shown to potently inhibit autoimmune responses and lead to immunological tolerance to the encephalitogenic MOG-35-55 peptide and reverse clinical and histological signs of EAE (Vandenbark et al., J. Immunol. 171:127-33, 2003). Numerous additional RTL constructs that are useful for modulating T cell immune responses and can be employed within the invention are available for use within the methods and compositions of the invention (see, e.g., U.S. Pat. No. 5,270,772, issued Aug. 7, 2001; U.S. Provisional Patent Application No. 60/064,552, filed Sep. 16, 1997; U.S. Provisional Patent Application No. 60/064,555, filed Sep. 16, 1997; U.S. Provisional Patent Application No. 60/200,942, filed May 1, 2000; United States Provisional Patent Application entitled MONOMERIC RECOMBINANT MHC MOLECULES USEFUL FOR MANIPULATION OF ANTIGEN-SPECIFIC T-CELLS, filed b_{y} Burrows et al. on Sep. 5, 2003 and identified by Attorney Docket No. 49321-98; U.S. patent application Ser. No. 09/153,586; filed May 1, 2001; U.S. patent application Ser. No. 09/847,172; filed May 1, 2001; and U.S. patent application Ser. No. 09/858,580; filed May 15, 2001, each incorporated herein by reference).

To evaluate the biological function and mechanisms of action of RTLs and other T cell modulatory agents, antigen-specific T cells bearing cognate TCRs have been used as target T cells for testing (see, e.g., Burrows et al., J. Immunol. 167:4386, 2001). However, a low frequency of Ag-specific T cells, varying levels of T cell Ag-specific responses, and a potential for uncontrolled interactions (e.g., with other, different cells) have significantly limited the scope of these investigations.

The T cell hybrids of the invention are uniquely adapted for use in screens and assays to identify and characterize RTL structure-function. To practice these aspects of the invention, T cell hybrids are constructed and selected that display an Ag-specific, TCR-mediated proliferative response contact of the hybrid with a cognate Ag and APCs. This proliferative response of T hybrids can in turn be detectably inhibited or stimulated by contacting the T cell hybrid with an RTL of interest, which yields a modified, Ag-specific, TCR-mediated proliferation response of the hybrid. The modified proliferation response of the hybrid cell accurately and reproducibly indicates a presence, quantity, and/or activity level of the RTL in contact with the T cell hybrid.

RTLs for use in these aspects of the invention will often comprise a MHC II component covalently linked or non-covalently bound to the cognate Ag. In exemplary embodiments, the RTL comprises a single chain MHC II component comprised of MHC II al and 131 domains. Alternatively, the RTL may comprise other single chain MHC II components which lack a β2 or other CD4-interactive domain. RTLs of interest in these aspects of the invention include RTLs which have the cognate Ag covalently linked or non-covalently bound to the MHC II component, wherein the cognate antigen comprises an immunodominant T cell epitope associated with a mammalian immune disorder, for example an autoimmune disease. In exemplary embodiments, the autoimmune disease is MS, and the immunodominant epitope is selected from human (hu)-myelin oligodendrocyte protein (MOG)-1-22, huMOG-35-55, huMOG-huMOG-1-22, huMOG-34-54, huMOG-63-87, huMOG-64-96, huMOG-92-106, murine (mu)-MOG-1-30, muMOG-35-55, muMOG-81-110, muMOG-91-110, rat (rt)-MOG-1-20, rtMOG-35-55, rtMOG-74-90, guinea pig (Gp)-myelin basic protein (MBP)-72-89, rt-MBP-72-89, hu-MBP-85-99, hu-MBP-86-99, hu-MBP-87-99, PLP-139-151. In more detailed embodiments, the RTL is VG1000, which includes a covalently bound hMOG-35-55 Ag.

In related embodiments of the invention, the RTL is modified to reduce higher order aggregate formation by the RTL. Such modifications include mutations in a MHC component of the RTL, for example by recombinant introduction of one or more mutations within a B sheet platform of an MHC II component.

In addition to Ag-specific TCRs and MHCs, a number of other regulatory molecules regulate T cell activation and impart sensitivity and plasticity to the immune response. Within the methods and compositions of the invention, it will be additionally useful to express one or more such regulatory molecules within the starting T cells, mammalian fusion partner cells, and/or the T cell hybrids described herein. In certain embodiments of the invention, the starting T cell, mammalian fusion partner cell, and/or the T cell hybrid is transgenically modified, transduced, or transfected to express an immune modulatory molecule. Often, the immune modulatory molecule will be a cytokine, for example IL-2. Another immune modulatory molecule of interest is the product of Fox P3, which may be expressed in the starting T cell or hybrid to drive a T suppressor phenotype or function in the hybrid cell.

In additional embodiments, the immune modulatory molecule is a T cell costimulation regulatory molecule, or an agonist, antagonist, or ligand of a T cell costimulation regulatory molecule. Exemplary modulatory molecules in this context include, OX 40, OX 40 ligand, CD 40, CD 40 ligand, CD 28, CD 28 ligand, CTLA4, and CTLA4 ligand.

In yet additional embodiments of the invention, the starting T cell, the mammalian fusion partner cell, and/or the T cell hybrid is transgenically modified, transduced, or transfected to express a neuroprotective or neuroregenerative factor. Exemplary neuroprotective or neuroregenerative factors in this context include nerve growth factor (NGF) and brain-derived neural factor (BDNF).

In still other embodiments, he T cell, the mammalian fusion partner cell, and/or the T cell hybrid is transgenically modified, transduced, or transfected to express a growth factor, for example IL-7 or IL-15.

The T cell hybrids of the invention are particularly well suited for screening, designing, and characterizing T cell modulatory agents for development as diagnostic and/or therapeutic agents in the management and treatment of multiple sclerosis (MS). MS is a chronic autoimmune disease characterized by recurrent attacks of neurologic dysfunction due to lesions in the central nervous system. MS lesions are marked by infiltration of inflammatory cells, such as lymphocytes, macrophages and neutrophils, and associated axonal demyelination (Martin et al., Crit. Rev. Clin. Lab. Sci. 32:121-82, 1995; Steinman, L., Nat. Immunol. 2:762-65, 2000). The classic clinical features of multiple sclerosis include impaired vision and weakness or paralysis of one or more limbs. After a number of years, patients generally experience a progressive deterioration of neurologic function. The disease course is unpredictable and involves exacerbations and remissions in 75% of patients. Although a few patients die within the first few years of onset, the average duration of the disease is greater than 30 years.

There are an estimated 250,000 cases of multiple sclerosis in the United States, with approximately 10,000 new cases occurring each year. The cause of MS is unknown but epidemiology implicates immunologic or infectious factors resulting in a chronic inflammatory condition. Multiple sclerosis is typically a disease of young adults, with more than 65% of cases initiating at ages 20-40. 60% of MS patients are women. Over one million physician visits occur annually for multiple sclerosis in the United States alone.

There is currently no effective treatment or cure for multiple sclerosis. MS therapy is directed toward reducing the severity of acute episodes and preventing relapses. In acute flare-ups, steroids are often employed to reduce severity and speed recovery. Experimental therapy with other immunosuppressive agents, such as cyclophosphamide, has also been attempted, but with limited success.

Both genetic and environmental factors have been implicated in the MS disease process. Genetic predisposition to MS includes an association of the disease with certain class II major histocompatibility complex (MHC) haplotypes, in particular HLA-DR2 (DRB1*1501) and DQw1 (Ho et al., Immunogenetics 15:509-17, 1982; Hauser et al., Neurology 39:275-77, 1989; Zipp et al., Hum. Immunol. 61:1021-30, 2000).

A widely accepted disease model for human MS is experimental allergic encephalomyelitis (EAE), an acute inflammatory and demyelinating disease of the central nervous system (CNS) bearing substantial clinical and pathological similarities to MS (Traugott et al., Science 219:308-10, 1983; Zamvil et al., Nature (London) 317:355-58, 1985; Rose, et al., Clin. Immunol. Immunopathol. 59:1-15, 1991. For both EAE and MS, there is considerable evidence that immunological and inflammatory processes contribute to the pathogenesis of the disease. (Hauser, et al., Ann Neurol. 13:418-25, 1983; Traugott, et al., Cell. Immunol. 68:261-75, 1982; Rose, et al., Clin. Immunol. Immunopathol. 45:405-23, 1987). This is supported by the presence of perivascular mononuclear cellular infiltrates in MS lesions and macrophage-dependent phagocytosis of myelin in the CNS white matter. (Prineas, et al., Lab. Invest. 38:409-21, 1978; Alvord, J C Koetsier, editor; Handbook of Clinical Neurol. 3(47):467-502, Koetsier (ed.); Amsterdam; Elsevier Science Publishers BV, 1985).

Recently, HLA-DR2+transgenic (Tg) mice have been developed that are susceptible to EAE induced by myelin basic protein (MBP)-85-99 peptide (Madsen et al., Proc. Natl. Acad. Sci. USA 96:10338-43, 1999 (1); Madsen et al., Nat. Genet. 23:343-47, 1999 (2)) and proteolipid protein (PLP) peptide 95-116 (Kawamura et al., J. Clin. Invest. 105:977-84, 2000). Myelin oligodendrocyte glycoprotein (MUG) appears to be recognized frequently by T cells from MS patients (KerlerodeRosbo et al., J. Clin. Invest. 92:2602-08, 1993; KerlerodeRosbo et al., J. Autoimmun. 11:287-95, 1998), and the MOG-35-55 peptide has been found to be highly encephalitogenic in rats and mice (Adelmann et al., J. Neuroimmunol. 63:17-27, 1995; Mendel et al., Eur. J. Immunol. 25:1951-59, 1995; Johns et al., J. Immunol. 154:5536-41, 1995). It has also been determined that MOG-specific TCR-Tg mice develop spontaneous autoimmune optic neuritis, another form of autoimmune demyelination (Bettelli et al., J. Exp. Med. 197:1073-81, 2003). MOG-35-55 peptide can induce severe chronic EAE in HLA-DRB1*1501-Tg mice (Rich et al., Eur. J. Immunol. 34:1251-61, 2004). A novel single-chain recombinant TCR ligand (RTL) comprised of the α1 and β1 domains of DRB1*1501 covalently linked to MOG-35-55 peptide can reverse clinical signs of MOG peptide-induced EAE (Vandenbark et al., J. Immunol. 171:127-33, 2003). Other reports have identified the region between amino acids 85 to 99 of myelin basic protein (MBP) as containing an immunodominant epitope for T cells and autoantibodies in MS brain lesions. The main region of MBP recognized by T cells and autoantibodies, found in MS brain, is reported to be a core motif, HFFK, from MBPp87-99 in patients who are HLA DRB1*1501 DQB1*0602 (HLA DR2) (see, U.S. Pat. No. 6,531,130, issued Mar. 11, 2003). Other MBP peptides of interest within the invention include a 10-amino acid segment 86-95 (VVHFFKNIVT) that also contains the MHC-T cell receptor contact residues for T cells recognizing MBP in the context of DRB1*1501 and DQB1*0602. In the epitope center, the residues VHFFK are reported to be important for T cell binding and MHC recognition. Recently, the crystal structure of HLA-DR2 with MBP 85-99 was solved, confirming the prediction that K91 is the major TCR contact residue, while F90 is a major anchor into the hydrophobic P4 pocket of the MHC molecule.

As described herein, above, the T cell hybrids of the invention can be generate to exhibit an Ag-specific, TCR-mediated activation response to any cognate Ag of interest, including the foregoing immunodominant epitopes identified in association with MS. By stimulating the T cell hybrids of the invention with these cognate Ags in the context of APCs, an activation response marked by discernable proliferation of the hybrid cells is elicited. This activation response, can be specifically inhibited or enhanced by TCR antagonists or agonists, which provides the basis for a wide range of assays to screen, identify, design, test and characterize novel TCR antagonists or agonists.

In one screening protocol of the invention, The T cell hybrid is characterized, as above, by its capacity to exhibit an Ag-specific, TCR-mediated proliferative response following contact with the cognate Ag and APC. The T hybrid is cultured under suitable conditions, and at a selected point in culture is exposed to a known or prospective TCR agonist or antagonist (which is a test compound or agent in the assay).

The test compound or agent can, for example, be a RTL as described herein. RTLs can comprise a MHC II component covalently bound to a cognate Ag that is an immunodominant epitope associated with a selected autoimmune disease, for example MS. The assay can be used to sensitively measure the effect of the RTL on Ag-specific activation of the T cell hybrid, which can be measured by comparing the Ag-stimulated proliferation response of the hybrid exposed to the RTL before, or after, Ag-stimulation, and comparing this proliferation to that of the same T hybrid under similar conditions but not exposed to the RTL.

As described in the examples below, certain RTLs will exert a detectable, inhibitory effect on the Ag-specific proliferative response of the T hybrid. This effect will be accurately and sensitively detectable in the assay methods and compositions of the invention, and may be paralleled by other activation changes in the T hybrid, for example by an increase in IL-2 production measured using an IL-2-dependent (e.g., CTLL) cell line. This assay design allows for screening and optimizing RTLs, including rational design of RTLs having directed structural changes in either the MHC component or Ag component of the RTL

Comparable assays are provided using the T hybrids of the invention to screen for many different classes of T cell modulatory agents. These modulatory agents may include agents that bind TCRs or otherwise interfere with TCR activity. Alternatively, the modulatory agents may bind or interfere with a T cell coreceptor, an MHC molecule of an APC or RTL, or a cognate Ag of a TCR associated with T cell regulation. In one exemplary assay, the T hybrids are used for screening or rational design of peptide or small molecule mimetics of important structural domains or motifs of TCRs, MHC molecules, and/or T cell Ags. In the case of a TCR mimetic, for example, a test compound may structurally mimic a portion of a TCR that interacts with Ag, for example an Ag-binding or Ag-interactive domain of the TCR. Desired TCR mimetics in this context will include peptides having an amino acid sequence that mimics (i.e., by exact or conservative sequence identity) a functional motif of the TCR and thereby is capable of binding, chelating, sequestering, inactivating, or otherwise impairing TCR-interactions by a TCR ligand or regulatory molecule (e.g., by blocking cognate Ag binding or APC costimulation) of the TCR. The effect of the test agent in this context can be detected by observation of an inhibitory or stimulatory effect on T hybrid proliferation and/or associated TCR-mediated responses in the presence of the test agent. If the T cell hybrid is contacted with the test agent, and subsequently or previously subjected to an Ag-specific stimulation (e.g., Ag plus APC, or by exposure to an RTL that activates or represses the T cell without APCs), the agonistic or antagonistic effect of the test agent will be indicated by an increase or decrease in proliferation by the hybrid.

Comparable assays are provided that screen, identify and/or characterize test compounds that structurally mimic a portion of a cognate Ag that interacts with the TCR, for example a TCR-binding or other interactive domain of the peptide. Desired peptide mimetics in this context, for example, will include peptides having an amino acid sequence that is altered from a native sequence (i.e., a naturally occurring, wild-type or mutant sequence) of a T cell epitope, most often a T cell epitope associated with an immune disorder. The test peptide may thereby exhibit modified TCR-binding and/or TCR-activation kinetics, which can be detected by observing inhibitory or stimulatory effects on T hybrid proliferation (and/or IL-2 production, etc.). If the T cell hybrid is contacted with the test peptide agent, and subsequently or previously subjected to an Ag-specific stimulation (e.g., Ag plus APC, or by exposure to an RTL that activates or represses the T cell without APCs), the agonistic or antagonistic effect of the test peptide will correlate with an increase or decrease in proliferation by the hybrid.

Various other assays are contemplated that incorporate the novel T hybrids of the invention in known assay formats. These include high throughput assays designed to detect modulators of T cell activity, TCR activity, MHC activity, and Ag effects on T cell biology, among other targets.

The T hybrids and assays of the invention are useful in developing diagnostic and therapeutic methods and compositions for MS, and also for a variety of other autoimmune disorders. Key targets for constructing the hybrids and designing the subject assays include TCRs, MHC molecules, and cognate Ags associates with the subject immune disorder. Another important autoimmune disease in this context is rheumatoid arthritis (RA). RA is an autoimmune disease that primarily affects peripheral joints with cartilage destruction and subsequent bone erosion. In a significant fraction of patients, this persistent synovitis leads to destruction of articular cartilage and surrounding structures and is a cause of significant morbidity. Importantly, the major genetic contribution to RA involves HLA class II alleles dominated by HLA-DRB1*0401 and *0404 (in DR4) and DRB1*0101 (in DR1) in Caucasian populations. These alleles all share a sequence motif at positions 67-74 of the third hypervariable region of the DRβ chain, termed the shared epitope (Gregerson et al., Arthritis Rheum. 30:1205-13, 1987), which profoundly affects peptide binding and CD4+T cell recognition (Nepom, Adv. Immunol. 68:315-32, 1998). Synovial tissue and fluid from inflamed joints of RA patients usually contain large numbers of CD4+ T cells, including activated CD4+ T cells. Studies of synovial T cells also have demonstrated sets of related oligoclonal CD4⁺ T cell expansions in individual patients that express highly homologous TCRs (Striegich et al., J. Immunol. 161:4428-36, 1998). Based on these findings, it has been hypothesized that disease-associated HLA-DR molecules present arthritogenic cartilage antigens and cause stimulation and expansion of antigen-specific T cells in the joint. This T cell response then drives the inflammatory process.

Previous studies have suggested that type II collagen (CII) and human cartilage gp39 (HCgp39) are among the most likely synovial antigens to be involved in T cell stimulation in RA. CII, the main constituent of hyaline cartilage, has been proposed as an important autoantigen in RA because CII-specific antibodies are frequently found in RA patients and because an RA-like disease can be induced in certain murine strains after immunization with CII. Studies performed in DR4- and DR1-expressing mice have located an immunodominant T cell epitope to position 263-270 in CII by using synthetic peptides (Fugger et al., Eur. J. Immunol. 26:928-33, 1996; Rosloniec et al., J. Immunol. 160:2573-78, 1998; Andersson et al., Proc. Natl. Acad. Sci. USA 95:7574-79, 1998). In later studies, Backlund and coworkers (Proc. Nat. Acad. Sci. USA 99:9960-65, 2002) used a humanized mouse model expressing HLA-DRB1*0401/DRA1*0101, human CD4, and human CII (huCII) on a background deficient of murine class II expression (Fugger et al., Proc. Natl. Acad. Sci. USA 91:6151-55, 1994; Malmstrom et al., Scand. J. Immunol. 45:670 77, 1997) to further elucidate the role and behavior of T cells in RA. From these studies the authors reported a dominant T cell response to glycosylated CII-glycopeptides in a cohort of severely affected RA-patients.

Further experiments have defined dominant peptide determinants of CII and gp39 antigens when presented by HLA-DR4, the most important RA-associated HLA type. Kotzin et al., (Proc. Nat. Acad. Sci. USA 97:291-96, 2000) used fluorescent, soluble peptide-DR4 complexes (tetramers) to detect synovial CD4+T cells reactive with CII and HCgp39 in DR4+ patients. The CII-DR4 complex bound in a specific manner to CII peptide-reactive T cell hybrids, but did not stain a detectable fraction of synovial CD4+ cells. A background percentage of positive cells (<0.2%) was not greater in DR4 (DRB1*0401) patients compared with those without this disease-associated allele. Similar results were obtained with the gp39-DR4 complex for nearly all RA patients. In a small subset of DR4+ patients, however, the percentage of synovial CD4+ cells binding this complex was above background and could not be attributed to nonspecific binding. These studies demonstrate the potential for peptide-MHC class II tetramers to be used to track antigen-specific T cells in human autoimmune diseases. Together, the results also suggest that the major oligoclonal CD4+ T cell expansions present in RA joints are not specific for the dominant CII and HC 39 determinants.

The T hybrids and related assays and compositions of the invention are also adapted for scientific and clinical investigation and management of type 1 diabetes, or IDDM. IDDM results from the destruction of pancreatic islet β cells by a complex autoimmune process to which both genetic and environmental factors appear to contribute. In humans, as in the accepted model for the human disease, NOD mice, the main genetic contribution to susceptibility resides in class II loci of the MHC, with particular sequences and structures in haplotypes that confer susceptibility in NOD mice and in diabetic human patients (Hattori et al., Science 231:733-35, 1986; Acha-Orbea et al., Proc. Natl. Acad. Sci. USA 84:2435 39, 1987; Corper et al., Science 288:505-11, 2000; Latek et al., Immunity 12:699 710, 2000; and Lee et al., Nat. Immunol. 2:501-07, 2001). In particular, IDDM is genetically associated with specific alleles from the HLA-DQ and HLA-DR loci shown to contribute to the disease. Among DR4 subtypes, HLA-DRB1*0401, HLA-DRB1*0402, and HLA-DRB1*0405 alleles lend susceptibility, while HLA-DRB1*0403 confers protection. In addition, glutamic acid decarboxylase isoform 2 (GAD65) has been identified as a key target autoantigen of IDDM.

IDDM is marked by the presence and activation of autoreactive T cells. Transgenic models have shown that, for the most part, T cells reactive against peripheral antigens are selected in the thymus much like other T cells are (Katz et al., Cell 74:1089-100, 1993; Lafaille et al., Cell 78:399-408, 1994; Goverman et al., Cell 72:551-60, 1993; and Verdaguer et al., J. Exp. Med. 186:1663-76, 1997).

Several autoreactive T cell clones isolated from diabetic NOD mice have been shown to be pathogenic in transfer experiments (Pankewycz et al., Eur. J. Immunol. 21:873-79, 1991; Daniel et al., Eur. J. Immunol. 25:1056-62, 1995); Haskins et al., Science 249:1433-36, 1990). BDC-2.5 is the best characterized of these clones, and has been shown to greatly accelerate disease when transferred to young animals. This CD4+ Th1 clone displays a Vβ4/Vα1 (AV1S5) TCR heterodimer, and is restricted by Ag7, the lone MHC class II molecule of NOD mice (Candeias et al., Proc. Natl. Acad. Sci. USA 88:6167-70, 1991; Acha Orbea et al., Proc. Natl. Acad. Sci. USA 84:2435-39, 1987). The nature of the presented antigen is still unknown, although it has been shown to be associated with the membrane fraction of β granules (Bergman et al., Diabetes 43:197-203, 1994). Mice transgenic for the BDC-2.5 TCR show a robust positive selection of CD4+ cells in the thymus. These are then exported, naive and fully reactive, to peripheral lymphoid organs and later infiltrate the pancreatic islets in a precocious and synchronized manner (Katz et al., Cell 74:1089-100, 1993).

As noted above, Patel and coworkers (Proc. Natl. Acad. Sci. USA 94:8082-87, 1997) utilized HLA-DR (DR0401), human CD4, murine class II null triple transgenic mice and recombinant GAD65 to generate T cell hybrids, and made overlapping sets of peptides to map the immunodominant epitopes of the GAD65 autoantigen. These authors identified 10 immunogenic regions for GAD65. These epitopes were also reportedly generated by human antigen-presenting cells, and their presentation is DR0401 restricted (as shown by the use of typed human lymphoblastoid cell lines and antibody blocking experiments). Immunodominant GAD65 epitopes defined in transgenic mice correspond to GAD65 regions previously shown to elicit T cell responses specifically in DR0401 IDDM patients. Although the major epitopes contain DR0401 binding motifs, one of the epitopes contains a DR0405 motif Additional immunogenic epitopes for use within the invention have been identified by Wicker et al., (J. Clin Invest. 98:2597-603, 1997), Lohmann et al., (Lancet 343:1607-08, 1994), and Lohmann et al., (J. Autoimmun. 9:385-89, 1996).

From these studies, exemplary IDDM-associated candidate peptides for use in generating hybrids and related compositions and methods within the invention include, but are not limited to: LYNIIKNREG; YNIIKNREG; LYNIIKNRE; LIAFTSEHS; FTSEHSHFS; FFRMVISNPAA; FRMVISNPA; and SLRTLEDNEER.

The T hybrids and related assays and compositions of the invention are also adapted for scientific and clinical investigation and management of psoriasis and other skin diseases known to exhibit autoimmune etiology. Onset of psoriasis may be triggered by systemic infections such as strep throat, skin injury, vaccinations, and certain oral medications such as steroids. Subsequently, the immune system is thought to induce inflammation and excessive skin cell reproduction, which can be exacerbated by additional factors such as stress and diet.

Previous reports have described a monoclonal antibody designated UM4D4 which recognizes the cell surface marker CDw60. This marker is present on a subset of normal T cells, melanocytes, malignant melanoma cells, and hyperproliferative psoriatic keratinocytes. CDw60 antibodies bind to the acetylated form of GD3. 74% of basal cell carcinomas express CDw60, whereas CDw50 expression in normal skin is confined to melanocytes and a few scattered keratinocytes at the basal cell layer. Psoriatic skin, basal and suprabasal keratinocytes all express CDw60. Cloned T cell lines obtained from lesional skin upon initiation have been shown to release soluble factors including IL-4 and IL-13, that up-regulated CDw60 expression on cultured normal keratinocytes. Consequently, CDw60 and other markers associated with psoriasis and other immune disorders of the skin also serves as targets and test agents within the methods and compositions of the invention.

The T hybrids and related assays and compositions of the invention are also adapted for scientific and clinical investigation and management of transplant complications, including graft-versus-host-disease (GVHD). Specific suppression of a host's immune response to donor HLA antigens remains the ultimate goal for clinical transplantation. In spite of considerable effort, however, allospecific human suppressor T cells (Ts) have been difficult to generate. Previous studies have shown that allospecific and xenospecific T suppressor cells can be raised by multiple priming of T cells in mixed lymphocyte cultures (MLC). Allospecific T suppressor cells prevent the upregulation of B7 molecules on target APCs, interfering with the CD28-B7 interaction required for T helper cell activation.

Transplant tolerance has been induced in adult animals by inactivation or depletion of mature T lymphocytes prior to transplantation using cyclosporine (CsA), total lymphoid irradiation, anti-lymphocyte serum, antibodies against CD4+ and CD8+ T cells, or donor-specific transfusions. Studies of peripheral graft tolerance have suggested the existence of an active mechanism of suppression which is donor-specific and can be transferred adoptively to secondary hosts. However, there is still controversy concerning the phenotypic characteristics of these regulatory T cells and their MHC restriction, as both CD8+ and CD4+ T cells have been reported to display suppressive activity. It has been suggested that suppression may result from antagonistic effects of (Th)2-type lymphokines (such as IL-4 and IL-10) on the response of T helper cells, or from recognition by T suppressor cells of either idiotypic determinants of the TCR of alloreactive T cells or of MHC antigens expressed on stimulating cells. The generation of T suppressor lines has proven, however, to be a difficult task rendering the characterization of these cells hard to achieve.

Xenospecific T suppressor cells prevent the up-regulation of CD154 molecules on the membrane of T helper (Th) cells, inhibiting their ability to react against the immunizing MHC-class II xenoantigens. The mechanism of this suppression, therefore, appears to be blockade of CD154/CD40 interaction required for efficient costimulation of activated T cells. Additional studies have shown that T suppressor cell lines can be generated by in vitro immunization of human PBMCs with synthetic peptides or soluble proteins coupled to beads. Such CD8+ T suppressor cells exhibit antigen specificity and restriction by self MHC class I molecules, limited TCR V beta gene usage, ability to inhibit antigen-specific, MHC Class II restricted, T helper cell proliferative responses, and capacity to downregulate and/or inhibit the upregulation by T helper cells of CD40, CD80, and CD86 molecules on APCs. These findings provide a basis for the development of specific immunosuppressive tools and therapies targeting regulation of immune responses in graft rejection using the T hybrid cells and assay methods of the invention as described herein.

RTLs and other compositions provided or identified using the compositions and methods of the invention are useful to treat individuals suffering from immune disorders, including demyelinating autoimmune diseases. Diagnosis of MS patients may utilize a variety of criteria known to those of skill in the art. A quantitative increase in myelin autoreactive T cells with the capacity to secrete IFN-γ is associated with the pathogenesis of MS and EAE. During the presymptomatic period there is infiltration of leukocytes into the cerebrospinal fluid, inflammation and demyelination. Family histories and the presence of the HLA haplotype DRB1*1501, DQA1*0102, DQB1*0602 are indicative of a susceptibility to the disease. Treatment during the early stages of the disease is preferred, in order to slow down or arrest the further loss of neural function.

Patients are diagnosed as having MS according to conventional clinical criteria. Such criteria rely on the presence of two attacks at least one month apart, where an attack is a sudden appearance of or worsening of an MS symptom or symptoms which lasts at least 24 hours; and more than one area of damage to central nervous system myelin. The damage to myelin must have occurred at more than one point in time and not have been caused by any other disease that can cause demyelination or similar neurologic symptoms.

MRI (magnetic resonance imaging) is the preferred method of imaging the brain to detect the presence of plaques or scarring caused by MS, although CT scans may also be used. Other symptoms include disability in mental, emotional, and language functions, movement and coordination, vision, balance, and the functions of the five senses. Evoked potential tests are electrical diagnostic studies which can show if there is a slowing of messages in the various parts of the brain, and may provide evidence of scarring along nerve pathways that is not apparent on a neurologic exam. Cerebrospinal fluid, usually taken by a spinal tap, may be tested for levels of cytokines, and for the presence of oligoclonal antibody band.

The therapeutic effect of compositions provided or identified by implementation of this disclosure may be measured in terms of clinical outcome, or may rely on immunological or biochemical tests. Suppression of the deleterious T cell activity can be measured by enumeration of myelin-reactive Th1 cells in spinal fluid, by quantitating the release of cytokines at the sites of lesions, or using other assays for the presence of autoimmune T cells known in the art. Alternatively, one may look for a reduction in symptoms of a disease, such as the damage to neural tissue observed in MS, or the decrease in-the number or severity of attacks of MS suffered by MS patients. Damage to neural tissue can be assessed for example by magnetic resonance imaging (MRI) and measurement of the number and severity of lesions visible therein. Reduction in MS attack number or severity can be assessed for example by clinical evaluation of patients. Methods for both MRI and clinical evaluation are well-known in the art.

Various methods for administration immune modulatory agents of the invention may be employed. The agents may be administered orally or injected, e.g. by intravascular, intratumor, subcutaneous, intraperitoneal, intramuscular, injection. The dosage of therapeutic formulations comprising the immune modulatory agents will vary widely, depending upon the nature of the disease, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered daily, semi-weekly, etc. to maintain an effective dosage level. In many cases, oral administration will require a higher dose than if administered intravenously.

Therapeutic compositions of the invention may also contain other therapeutically active agents, e.g. immunosuppressants, β-interferon, steroids, etc. Of particular interest are combinations with other agents capable of additive or synergistic effect in achieving a therapeutic result, e.g. where a different or complementary pathway is affected by each of the active agents. Immunosuppressants of interest include cyclosporins A and G, FK-506, mycophenylate mofetil, rapamycin, azathioprine, antibodies for plasma membrane proteins associated with graft rejection, such as antibodies to CD4, CD8, CD2, LFA-1, ICAM-1, CD28, and the like; and immunosuppressive oligopeptides derived from MHC molecules. Antibacterial, antiviral and antifungal drugs may also be co-formulated in order to minimize the effects of immunosuppression.

Those of skill will readily appreciate that dose levels can vary as a function of the specific compound, the severity of the symptoms and the susceptibility of the subject to side effects. Some of the specific peptides are more potent than others. Preferred dosages for a given immune modulatory agent are readily determinable by those of skill in the art according to the teachings herein supplemented by knowledge in the art.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the methods and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g., amounts, temperature, concentrations, etc.) but some experimental errors and deviations should be allowed for.

### Examples

Within the following examples, BW5147 variant cells were used as fusion partners to generated CD4+ T cell hybrids that strongly respond to both human and mouse MOG-35-55 peptides and uniquely express TCR BV8 in conjunction with AV17 and AV11 for the recognition of human MOG-35-55 peptide restricted by the HLA-DR2/DRB1*1501 allele. Additionally, these examples show that MOG peptide-specific proliferation and cytokine responses of these T cell hybrids can be inhibited by a specific RTL containing HLA-DR2 external domains covalently linked to the cognate human MOG-35-55 peptide. This reduction of activation of the T cell hybrids in vitro by the RTL was reflected by the RTL's ability to inhibit EAE induced by human MOG-35-55 in DR2-Tg mice. These data demonstrate the powerful utility of peptide-specific T hybrid cells as a standardized biological screening tool.

### DR2 Transgenic Mice

HLA-DR2 (DRB1*1501 and DRB1*1502) mice used herein have been described previously (Rich et al., Eur. J. Immunol. 34:1251-61, 2004; Gonzalez-Gay et al., Hum. Immunol. 50:54-60, 1996). The mice were bred and housed at the Portland Veterans Affairs Medical Center, Portland, Oreg., under pathogen-free conditions according to institutional guidelines. Offspring were screened by flow cytometry by examining transgene expression on APC from whole blood obtained from the tail.

### Antigens

Mouse (m)MOG-35-55 peptide (MEVGWYRSPFSRVVHLYRNGK), human (h)MOG-35-55 peptide (MEVGWYRPPFSRVVHLYRNGK), MBP-85-99 peptide and mouse PLP-95-116 peptide were synthesized using solid-phase techniques and purified by HPLC at the Beckman Institute, Stanford University (Palo Alto, Calif.). Purified protein derivative (PPD) was purchased from the Staten Institute (Copenhagen, Denmark). Con A was purchased from Sigma-Aldrich (St. Louis, Mo.).

### MOG-35-55-Specific T Cell Lines

Tg mice were immunized with mMOG-35-55 or hMOG-35-55 peptides in CFA containing heat-killed Mycobacterium tuberculosis, strain H37RA (Difco, Detroit, Mich.). T cells were recovered from the spleen or draining lymph nodes of the Tg mice 10 days after immunization and cultured as cell lines in 2% fetal bovine serum (FBS)-containing RPMI 1640 medium supplemented with 0.05 mM 2-mercaptoethanol, 2 mM L-glutamine, 1 mM sodium pyruvate, 100 μg/ml penicillin G, 100 μg/ml streptomycin (GIBCO, New York) and 5 ng/ml r1L-2 (R & D Systems, Minneapolis, Minn.). These cell lines were re-stimulated with 10 μg/ml MOG-35-55 peptide in the presence of irradiated (2500 rad) syngeneic antigen-presenting cells (APC) for 2 days followed by culturing in rIL-2-containing medium for additional 3 days prior to cell fusion. Proliferation responses were assessed by using 4×10⁴ T cells plus 2×10⁵ syngeneic APC per well with 1% FBS-containing stimulation medium alone or in the presence of Ags at varied concentrations in triplicate. Cultures were incubated for 72 hr at 37 C in 7% CO₂ and pulsed for the final 18 hr with 0.5 μCi per well [³H]thymidine (Amersham, Arlington Heights, Ill.).

### BW5147 Tumor Cell Lines

AKR BW5147.G.1.4 cell line was purchased from the American Type Culture Collection (ATCC, Cat. #TIB 48) and cultured in 10% FBS-containing RPMI 1640 medium supplemented with 2 mM L-glutamine, 1 mM sodium pyruvate, 100 μg/ml penicillin G, 100 μg/ml streptomycin, and 0.1 mM non-essential amino acids (Invitrogen, Carlsbad, Calif.). The BW5147 variant cell line was obtained from the National Jewish Medical and Research Center, Denver, Colo. and cultured in the same medium as that used for BW5147.G.1.4 cell line.

### Cell Fusion and T Cell Hybrid Selection

Cell fusion was accomplished by using 50% polyethylene glycol 1500 (PEG 1500) (Roche Diagnostics, Mannheim, Germany) with procedures as recommended by the manufacturer. After fusion, the cells were cultured in selection medium, e.g. 10% FBS-containing RPMI 1640 culture medium supplemented with 1× hypoxanthine aminopterin thymidine (HAT)-media supplement (GIBCO), and distributed as 0.2 ml per well in a 96-well plate. After 2 weeks, unfused T cells gradually died off because of the lack of exogenous IL-2, and unfused HAT-sensitive BW tumor cells also died off, leaving only the dividing HAT-resistant T cell hybrids. T hybrid cells from each well were removed respectively to individual T25 tissue culture flasks (Costar, Corning, N.Y.) containing HAT-free medium supplemented with “Tumor Cocktail Star” supplied by the National Jewish Medical and Research Center (Denver, Colo.).

### Characterization of T Cell Hybrid

IL-2 bioassay: 10⁵ hybrid cells per well of a 96-well plate in 100 μl of 1% FBS-containing medium were combined with 10⁶ irradiated (2500 rad) syngeneic APC or irradiated (4500 rad) 4×10⁴ HLA-DRB1*1501-transfected 1 cells (Klohe et al., 1988)(a gift of Dr. R. Karr, Searle Research and Development, St. Louis, Mo.) in 100 μl alone or in the presence of 10 μg/ml of individual peptides or 5 μg/ml Con A, and incubated at 37 C and 7% CO₂ for 24 hr. Supernatants were collected from the top of the culture, followed by centrifugation for 1 min at 1000 rpm. Hybrid supernatants were added in triplicate at 50, 25, 10, or 2% into wells containing 5,000 CTLL-2 cells in 100 μl of 1% FBS culture medium. After 24 hr of culture, the cells were pulsed with 0.5 μCi [³H]Thymidine for an additional 6 hr. Stimulation index (SI) was calculated as the cpm of Ag-stimulated cultures divided by the cpm of the cells cultured in medium.

FACS analysis: Hybrids and BW tumor cells were stained with 4 μl of the following antibodies: anti-CD3-FITC, anti-CD4-PE, anti-CD8-FITC, and anti-mouse IgG1-PE (BD PharMingen, Calif.) for 30 min at 4 C. After washing 2×, the cells were analyzed using Cell Quest™ software on a FACScan. Hybrids were stimulated with 5 μg/ml Con A for 2 days in the presence of DRB1*1501-transfected L cells, and Brefeldin A was added for the last 6 hr followed by cell surface staining with anti-CD4-FITC. All surface-stained cells were washed 2× with p17.2 PBS, then fixed and permeabilized as defined by the manufacturer (BD PharMingen). Permeabilized cells were stained respectively with anti-IL-2-PB, anti-IFN-γ-PE and anti-TNF-α-PE. Anti:mouse TCR beta-Cy-Chrome and anti-AV11-FITC have been used for cell surface staining and intracellular staining.

### RT-PCR Analysis of TCR V Gene Expression

Relative AV and BV gene expression was analyzed by semi-quantitative PCR as previously described (Buenafe et al., J. Neurosci. Res. 47:489-99, 1997) with modifications. Briefly, cDNA was synthesized from total RNA with oligo(dT) and Superscript II reverse transcriptase (Life Technologies, Gaithersburg, Md.). BV-specific PCR products were generated using BV-specific primers and a fluorescent Tet-labeled BC primer with an application profile: 26 cycles at 94.5 C for 30 s, 55 C for 60 s, and 72 C for 60 s followed by a 5-min extension time at 72 C. AV-specific PCR products were similarly generated using AV primers and a fluorescent Cy3-labeled AC primer, mCab (Casanova et al., J. Exp. Med. 174:1371-83, 1991), using the same amplification profile. PCR products were separated on a polyacrylamide gel and the gels were scanned for the presence of fluorescent bands using a Bio-Rad Molecular Imager FX (Bio-Rad Laboratories, Hercules, Calif.).

### RTL Preparations

Single chain human RTLs containing approximately 200 amino acid residues derived from HLA-DR2b (DB1*1501) have been described previously (Chang et al., J. Bio. Chemistry 276:24170-76, 2001; Huan et al., J. Chem. Technol. & Biotechnol. in press, 2004). These RTLs were designed using the same principles as for rat RTLs and have been produced in Escherichia coli with and without amino-terminal extensions containing antigenic peptides as described previously (Burrows et al., Prot. Eng. 12:771-78, 1999; Chang et al., J. Bio. Chemistry 276:24170-76, 2001). Structural characterization using circular dichroism predicted that these molecules retained the antiparallel β-sheet platform and antiparallel α-helices observed in the native HLA-DR2 heterodimer. DR2-derived RTLs exhibited a cooperative two-state thermal unfolding transitions. When covalently linked to human MOG-35-55 or MBP-85-99 peptides, the RTLs showed increased stability to thermal unfolding relative to empty DR2-derived RTLs. To modify the β-sheet platform of the DR2-derived RTLs that formed higher order aggregations, a site-directed mutagenesis was performed to obtain a monomeric form of RTL which contained covalently tethered antigen peptides and capable of exhibiting its biological functions (Huan et al., J. Chem. Technol. & Biotechnol. in press, 2004). RTL VG1000 is a DR2-derived monomeric RTL covalently linked to human MOG-35-55 peptide (Virogenomics, Portland, Oreg.). RTL340 is also a DR2-derived monomeric RTL, but covalently linked to MBP-85-99 peptide produced in our laboratory. Both RTL VG 1000 and RTL340 are dissolved in 10 mM Tris buffer stock solution and aliquotted at a concentration of 40 uM and stored at −80 C.

### RTL In Vitro Potency Assay Using T Cell Hybrids

Human MOG-35-55 peptide-specific T cell hybrid cells (2×10⁵/well) were co-cultured in triplicate with 2 mM Tris-containing medium alone, 8 uM RTL VG1000 or 8 uM RTL340 in 2 mM Tris-containing medium respectively for 24, 48, 72 and 96 hr with uptake of [³H]Thymidine assessed for the last 6 hr before the end of each culturing time. The culture plates were harvested and the proliferation levels of cultured hybrid cells were calculated and compared by net cpm (mean±SD). Student's t test was used to assess significant differences (p<0.05). Aliquotted hybrid cell cultures were thoroughly washed with RPMI after 24 hr and 96 hr co-culturing and further stimulated with and without 10 μg/ml hMOG-35-55 peptide presented by irradiated (4500 rad) DRB1*1501-transfected cell lines at a 1:1 ratio in triplicate for 48 hr. Half of the supernatant was collected from top of each well and transferred into corresponding wells of another culture plate in which 100 μl of 1% FBS-containing medium with 5000 CTLL cells per well had been seeded. After 24 hr of culture, the CTLL cells were pulsed with [³H]Thymidine for an additional 6 hr. The net cpm (mean±SD) were calculated and compared between different conditions.

Induction of Active EAE and Treatment with RTL VG1000

Transgenic HLA-DR2 male and female mice between 8 and 12 weeks of age were immunized subcutaneously at four sites on the flanks with 0.2 ml of an emulsion comprised of 200 μg human MOG-35-55 peptide in CFA containing 400 μg heat-killed Mycobacterium tuberculosis H37RA (Difco, Detroit, Mich.) as described previously (Vandenbark et al., J. Immunol. 171:127-33, 2003). In addition, mice were given pertussis toxin (Ptx, List Biological Laboratories, Campbell, Calif.) on Days 0 and Day 2 post-immunization (75 ng and 200 ng per mouse, respectively). Mice were treated i.v. daily for 8 days, beginning on the second day after onset of clinical signs, with 100 μl of 1 mg/ml RTL VG1000 or vehicle (Tris, pH 8.5). Actively immunized mice were assessed daily for clinical signs of EAE according to the following scale: 0=normal; 1=limp tailor mild hindlimb weakness; 2=limp tail and moderate hindlimb weakness or mild ataxia; 3=limp tail and moderately severe hindlimb weakness; 4=limp tail and severe hindlimb weakness or mild forelimb weakness or moderate ataxia; 5=limp tail and paraplegia with no more than moderate forelimb weakness; and 6=limp tail and paraplegia with severe forelimb weakness or severe ataxia or moribund condition. The mean disease score and standard deviation were calculated on each day for the affected mice.

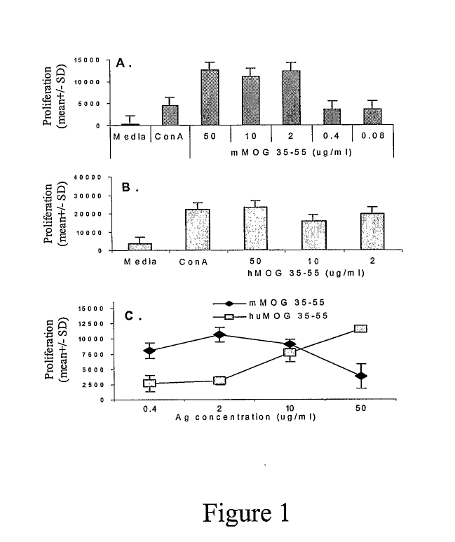

For generating MOG-35-55-specific T cell lines used for fusion, splenocytes or lymphocytes from draining from draining lymph nodes of HLA-DRB1*1501-Tg mice immunized with either mMOG-35-55 or hMOG-35-55 peptides were tested for Ag-specific responses. Two T cell lines responded strongly to either mMOG-35-55 or hMOG-35-55 peptide that was used initially for immunization, as shown in part A and B of FIG. 1. A T cell line selected from mMOG-35-55 peptide-immunized spleen cells had the same level of cross-recognition of hMOG-35-55 peptide at a concentration 10 μg/ml, but 3-fold lower responsiveness to hMOG-35-55 peptide at lower concentrations of 2 and 0.4 μg/ml, as is shown in FIG. 1C. This result suggests that a high functional TCR avidity was developed in recognition of the immunizing peptide antigen versus the cross-reactive peptide (Margulies, D. H., 2001). The human MOG-35-55-reactive lines were also cross-reactive to mouse MOG-35-55 peptide at the same responsive levels with the same Ag dose-dependent pattern.

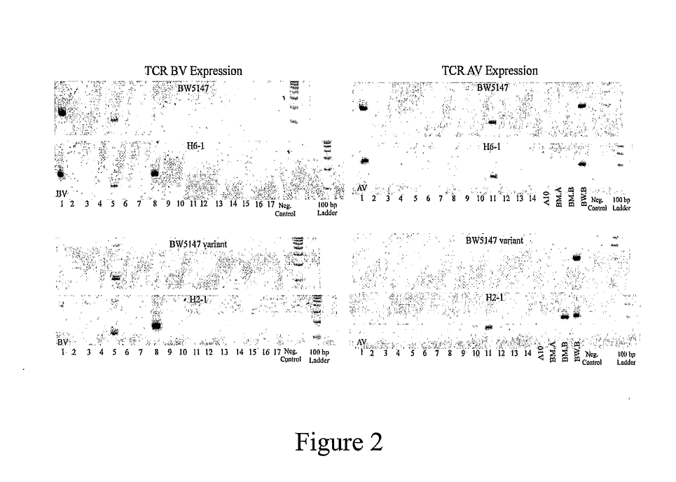

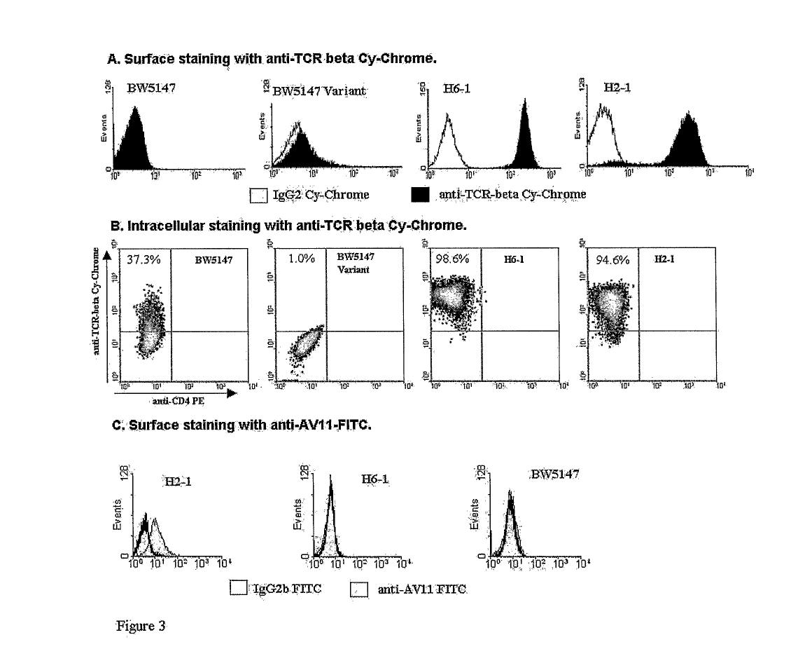

AKR BW5147.G.1.4 thymoma cells are resistant to 6-thioguanine and die in hypoxanthine aminopterin thymidine (HAT)-selective medium (Goldsby et al., Nature (London) 267:707-08, 1977). This fusion partner did not respond to MOG-35-55 peptides or PPD, and its response to Con A was slightly inhibited, consistent with previous reports (Indrova et al., Folia. Biol. (Praha) 30:390-95, 1984). Although BW5147 does not express surface TCR (Yague et al., Cell 42:81-87, 1985), isolation of cDNA clones from hybrids indicated that BW5147 had at least one functional α-chain and one functional β-chain rearrangement (Barth et al., Nature 316:517-23, 1985). BW5147 expressed BV1, BV5 and AV1, AV11 and AV16S1P (BW.B)(Letourneur et al., Eur. J. Immunol. 19:2269-74, 1989), as shown in FIG. 2 (top panel), which is consistent with previous reports (Chien et al., Nature 312:31-35, 1984; Blackman et al., Cell 47:349-57, 1986; Barth et al., Nature 316:517-23, 1985). BW5147 variant cells are also truly no longer expressing BV1, AV1 and AV11 as shown in FIG. 2 (third panel). This result is consistent with that originally demonstrated by an RNA dot blot hybridization analysis (White et al., J. Immunol. 143:1822-25, 1989). However, the BV5 and AV16S1P (BW.B) products were still present in these BW5147 variant cells. Neither BW5147 nor its variant were found to express TCR on the cell surface, as we confirmed by negative staining data shown in FIG. 3A. However, endogenous expression of TCR V genes by BW5147 was detected by positive intracellular staining of 37% of cells using anti-TCR beta-Cy-Chrome (FIG. 3B), whereas the B8W5147 variant had essentially no intracellular staining for TCR •• chains.

The TCR repertoire used in response to MOG peptides was next determined. According to prior reports, after MOG peptides were injected into C57BL/6J mice, the mice developed significant T cell responses to MOG peptides 1-21, 35-55 and 104-117. However, MOG-35-55 was the only MOG peptide that induced neurological impairment in C57BL/6J mice (Mendel et al., Eur. J. Immunol. 25:1951-59, 1995). The encephalitogenic MOG-35-55-reactive T cell lines predominantly expressed the BV8 gene product (40-43%) (Mendel et al., Eur. J. Immunol. 25:1951-59, 1995). The presently disclosed MOG-35-55-reactive T cell hybrids, H2-1 and H6-1, uniquely expressed BV8 as shown in FIG. 2 (Panel 2 and 4). The utilization of AV gene products by hybrid clones was also examined, which revealed that the clone H2-1, which was an hMOG-35-55-selected T cell fused with a BW5147 variant cell, expressed AV17S3 (BM.B) (Couez et al., Int. Immunol. 3:719-29, 1991; Ballenthin et al., J. Neurosci. Res. 46:271-81, 1996) and AV11 as shown in FIG. 2 (Panel 4). Clone H6-1, a mMOG-35-55-selected T cell fused with a BW5147 cell, expressed AV1, AV11 and AV16S1P (BW.B) as shown in FIG. 2, Panel 2. Because of the availability of anti-AV11-FITC (RR8-1) (BD PharMingen), the clones H2-1 and H6-1 and the BW5147 cells were stained with this Ab, which revealed a clear positive expression of AV11 on the cell surface of H2-1, but no detectable staining for AV11 on H6-1 or BW5147, as shown in FIG. 3C. These data suggest that AV11 is used for MOG-35-55-recognition by clone H2-1. However, AV11 in clone H6-1 is only expressed endogenously, apparently adopted from the fusion partner BW5147, and the significant AV gene used for Ag-recognition could not be otherwise identified. In future studies, we will evaluate additional AV gene primers by RT-PCR, as well as mAbs for other AV genes.

Interestingly, both clone H2-1 initially selected with hMOG-35-55 and clone H6-1 initially selected with mMOG-35-55 used BV8 in combination with a certain set of AV genes, in spite of the one amino acid difference at position 42 of the two MOG-35-55 sequences. This biological phenomenon may be due to the fact that this amino acid change (serine residue 42 of mouse MOG-35-55 changed to proline in the human sequence) does not dramatically alter interaction of these peptides to the DRB1*1501 binding pocket (Ballenthin et al., J. Neurosci. Res. 46:271-81, 1996), thus minimizing conformational changes that might affect selection of TCR V genes.

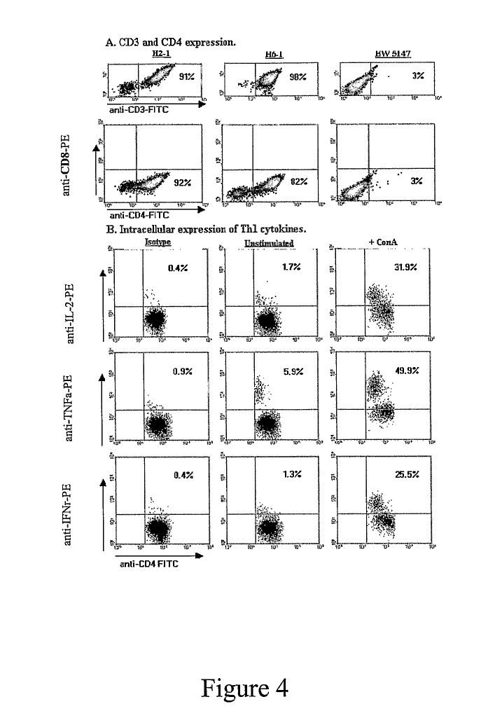

Phenotypically, T cell hybrids expressed CD3 and CD4 at levels greater than 90%, as is shown in FIG. 4A. The cytokine expression pattern showed that the CD4+ T cell hybrid clones had strongly enhanced expression of IL-2 (30%), TNF-α (44%), and IFN-γ (24%), but not IL-4 or IL-10, upon Con A stimulation for 48 hr (FIG. 4B). This result indicates that these T cell hybrid cells are Th1 type. Although BW5147 cells can suppress Th2 cytokine production (Weiss et al., 1997) and may influence the cytokine pattern of the hybrids, it is also evident that the Th1 type pattern expressed by hybrid clones is representative for MOG-35-55-specific T cells selected from the immunized mice, because the MOG-35-55-specific T cells used for the fusion showed a similar cytokine profile.

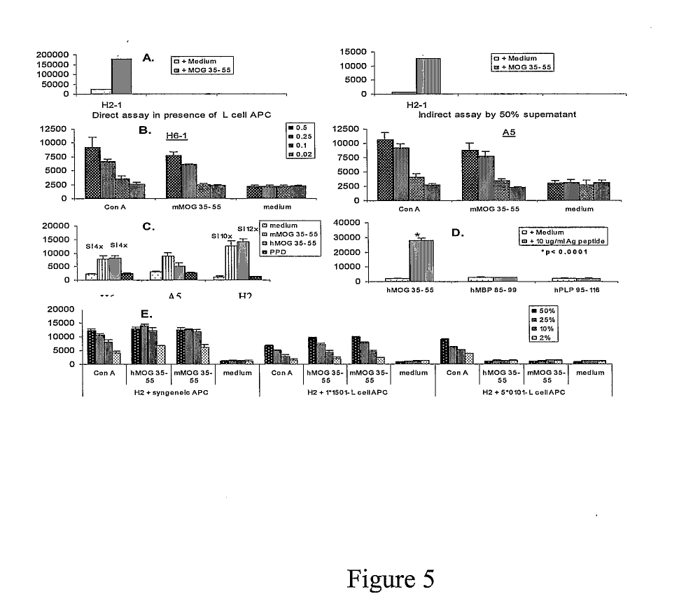

Functionally, clone H2-1 strongly responded to hMOG-35-55 peptide by proliferation in the presence of DR81*1501-transfected APC (direct assay) (SI 8×) and an indirect assay of culture supernatants on CTLL-2 cells (SI 18×), as shown in FIG. 5A. The hybrids H6 and AS, both generated from fusions between mMOG-35-55-specific T cells and BW5147 cells, showed strong responses to mMOG-35-55 and Con A (FIG. 58). Regarding cross-reactivity, the 1-12 hybrid was strongly cross-reactive with mMOG-35-55 (SI 10×) and the H6 responded equally well to both MOG-35-55 peptides at 10 μg/ml. The A5 was also cross-reactive with human MOG-35-55 peptide as is shown in FIG. 5C. Finally, the H2 hybrid was found not to respond to M8P-85-99, PLP-95-116, or PPO (FIG. 5D).

To identify MHC restriction molecules, three types of APCs were used to present Ag to the H2 hybrid. These APC were 1) syngeneic irradiated thymocytes with no expression of mouse MHC class II molecules but a 40% expression of HLA-DR; 2) DR81*1501-transfected L cells; and 3) DR85*0101-transfected L cells (Klohe et al., J. Immunol. 141:2158-63, 1988). As shown in FIG. 5E, the H2 hybrid responded best to MOG-35-55 peptides and Con A by using syngeneic APC. The DR81*1501-transfected APC were able to stimulate T cell responses to Ag and Con A up to 75% of the syngeneic APC levels. However, DR85*0101-transfected APC were not able to stimulate any Ag-specific T cell responses, but stimulated Con A response. These data clearly demonstrate DR81*1501 restriction of the MOG-35-55-stimulated T cell responses, consistent with the result from the systematic study of mapping T cell epitopes of MOG in HLA Tg mice by Chella David's laboratory (Khare et al., Int. Immunol. 15:535-46, 2003). The hybrid clone was stimulated with MOG-35-55, generating a cytokine (Th1) profile very similar to that induced by Con A stimulation (data not shown).

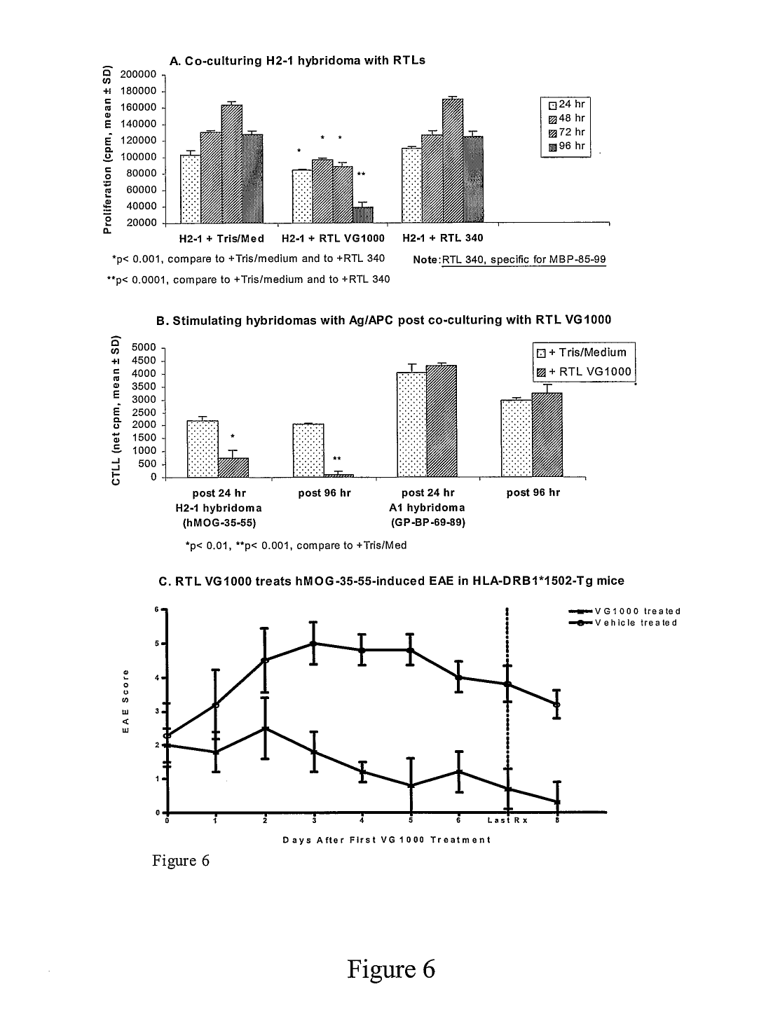

Having demonstrated specificity and MHC restriction, further investigations were conducted on the humanized T cell hybrids for testing inhibitory signaling properties of novel recombinant TCR ligands (RTLs), which have the capacity to inhibit EAE in vivo. In particular, an in vitro test was devised to assess the biological activity of VG1000 (external domains of DR2 linked to hMOG-35-55 peptide) that is a candidate for human therapy trials in MS. As is shown in FIG. 6A, constitutive proliferation responses of H2-1 hybrid cells co-cultured with RTL VG1000 for 24, 48, 72 or 96 hr all were significantly inhibited compared to the same cells co-cultured with medium containing identical amount of Tris buffer or RTL340 containing the same DR2 moiety but covalently linked to M8P-85-99 peptide. In a second type of assay, aliquots of H2-1 hybrid cells were co-cultured with RTL VG1000 or Tris-containing medium for 24 or 96 hr, collected and thoroughly washed with RPMI, and then restimulated with 10 μg/ml hMOG-35-55 peptide in the presence of DR81*1501-transfected L cells (A PC). After 48 hr, the supernatants were transferred into IL-2-dependent CTLL cell cultures for 24 hr and proliferation assessed by uptake of [³H]thymidine. As is shown in FIG. 6B, IL-2 secretion responses of the H2-1 hybrid cells to hMOG-35-55 peptide were inhibited 55% and 95% after respective 24 h or 96 h co-cultures with RTL VG1000 compared to co-culture in medium alone. To control for non-specific inhibition, RTL VG 1000 was co-cultured in an identical manner with the A1 T cell hybrid specific for GP-M8P-69-89 peptide (Gold et al., J. Exp. Med. 174:1467-76, 1991), and as is shown in FIG. 6B, no inhibition of the A 1 hybrid response to GP-M8P-69-89 peptide was detected after a 24 h or 96 h incubation with RTL VG1000. These data demonstrate that the humanized Ag-specific T cell hybrids generated by from the DR2 Tg mice can be used as an unlimited cell source to test in vitro biological functions of RTL preparations in an antigen-specific fashion.

To confirm that the inhibitory activity of RTL VG1000 preparation detected by the H2-1 hybrid could be related to its inhibitory activity in vivo, the ability of RTL VG1000 to reverse EAE in DR2 Tg mice was evaluated. Initially, hMOG-35-55 peptide was tested and shown to be both immunogenic and encephalitogenic in DRB1*1502 mice. The *1502 allele differs from *1501 by only a G for V substitution at position 86 in the P1 binding pocket, but this substitution did not affect immunogenicity of known DR2-restricted peptides from neuroantigens or recall antigens. Following immunization with hMOG-35-55 in CFA, moderately severe EAE (score of about 2) was induced in a total of nine *1502-Tg mice. After onset of clinical signs, 3 mice were treated with 100 ug RTL VG1000 given i.v. daily for 8 days, and 6 mice were treated under the same conditions with Tris buffer (vehicle). As is shown in FIG. 6C, the severity of EAE decreased to a score of 0.2±0.3 in mice treated with VG1000, whereas the severity of EAE increased to an average score of 4-5 in vehicle-treated control mice. These in vivo test data demonstrated the potent inhibitory effect of RTL VG 1000 on hMOG-35-55-induced EAE, and provided reference for the corresponding inhibitory effects of RTL VG1000 on the humanized hMOG-35-55 peptide-specific H2-1 hybrid.

Earlier studies showed that partial activation of a rat T cell hybrid by an Ag-specific RTL induced a TCRζ p23/p21 ratio shift, ZAP-70 phosphorylation, calcium mobilization, NFAT activation, and transient IL-2 production (Wang et al., J. Immunol. 171:1934-40, 2003). Similar characterization will be conducted for the MOG-35-55-specific T cell hybrids of the invention to assess signal transduction events that relate to the inhibitory functions of RTLs. The finding of paired TCR αβ genes will facilitate analysis of the sequences of both VDJβ and VJα1. (Cα.) regions to determine the specific motifs associated with autoimmune encephalitogenic activities (Buenafe et al., J. Immunol. 158:5472-83, 1997), and will allow a disclosure of the TCR sequences of specific genomic clones from our T cell hybrids. MOG-35-55 specific hybrids may also be valuable in screening and testing altered peptide ligands that could induce cytokine shifts in human T cell clones (Bielekova et al., Nat. Med. 6:1167-75, 2000).

Additional characterization studies were conducted to elucidate the starting T cell phenotype for optimal T cell hybrid construction. H2-1 and BW5147 variant tumor cells 1×10⁶ per each were stained with 5 μl of the following rat monoclonal antibodies specific for mouse CD44-Cy-Chrome (clone IM7), CD62L-FITC (clone MEL-14), and CD45RB-PE (clone 16A), and an additional 0.5×10⁶ cells per each were stained with 5 μl of hamster anti-mouse CD27-PE (clone LG.3A10) for 30 min at 40° C. Rat IgG2a-PE, Rat IgG2a-FITC, Rat IgG2b-Cy-Chrome, and Hamster IgG1-PE were used for isotype control staining. After washing 2× with PBS, the cells were analyzed using Cell Quest™ software on a FACScan. All monoclonal antibodies were purchased from BD PharminGen (San Diego, Calif.).

According to these and other studies, the exemplary T hybrid H2-1 was shown to have the following marker phenotype: CD45RB+, CD27+, CD62L+, CD44−

In additional work directed toward construction of human fusion partner cells for creating T hybrids, an HPRT deficient TCR-dependent Jurkat (J.RT3) mutant cell line was developed. This was initiated using a limited number of PBMC in primary peptide-stimulation followed by extended culture in rIL-2 to select T cell clones from a few HLA-DR2 homozygous MS patients. A suitable human T cell tumor line J.RT3 was chosen as a starting fusion partner because of the CD3- and TCR-deficient phenotype of this line. However, the original J.RT3 line was positive for HPRT, and therefore not useful as a fusion partner. The HPRT-mutant of this line is generated at a low rate by irradiation and growth in 6-thioguanine-containing medium, which kills un-mutated J.RT3 HPRT+cells.

For development of a J.RT3 derivative fusion partner, cell clones specific for MBP 85-99, PLP 139-151, MOG 35-55. All selected clones were shown Ag-specific proliferation with a distinct TCR expression pattern. The J.RT3 line was irradiated for 1 min and cultured with increased concentrations of 6-thioguanine (up to 20 ug/ml) for 3 months to obtain a J.RT3 HPRT minus mutant cell line. The mutant J.RT3 line we created has kept the same growth rate as the original tumor line and maintains its phenotype and no TCR cell surface expression same as the original J.RT3 line, but it can not survive in HAT-selecting medium as we expected!

A total of 21 mature hybrids were generated. All hybrids expressed low and varied amount of CD3 and TCR, though only in a few of them the CD3 was significantly higher than that expressed by J.RT3 mutants. The data show successful generation of human T cell hybrids from J.RT3 mutant fusion partners. Further development of the J.RT3 mutant fusion partners will lead to optimization of their use for generating human T cell hybrids having improved T cell phenotypes.

### Deposit of Biological Materials

The exemplary T cell hybrid H2-1, as described herein above, has been deposited with the American Type Culture Collection, (ATCC), Manasass, Va. and assigned Accession Number PTA-6082. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

It will be understood by those skilled in the art that the foregoing description is intended to illustrate and not limit the scope of the invention defined by the appended claims. Other aspects, advantages, and modifications will be appreciated as embodied within the scope of the invention.

What is claimed is:

1. A mammalian T cell hybrid comprising a fusion product of a mammalian T cell expressing a T cell receptor (TCR) and a mammalian fusion partner cell to yield a clonal T cell hybrid, wherein said T cell hybrid exhibits an antigen (Ag)-specific, TCR-mediated proliferative response following contact of the T cell hybrid with a cognate Ag and an allogeneic Ag-presenting cell (APC).

2. The T cell hybrid of claim 1, wherein the Ag-specific, TCR-mediated proliferative response of said T cell hybrid following contact with the cognate Ag and APC can be detectably inhibited or stimulated by contacting the T cell hybrid with a TCR antagonist or TCR agonist to yield a modified, Ag-specific, TCR-mediated proliferation response of the hybrid cell, wherein said modified proliferation response indicates a presence, quantity, and/or activity level of the T cell antagonist or T cell agonist in contact with the T cell hybrid.

3. The T cell hybrid of claim 1, wherein the Ag-specific, TCR-mediated proliferative response of said T cell hybrid following contact with the cognate Ag and APC can be detectably inhibited or stimulated by contacting the T cell hybrid with a recombinant T cell receptor ligand (RTL) to yield a modified, Ag-specific, TCR-mediated proliferation response of the hybrid, wherein said modified proliferation response of the hybrid cell accurately and reproducibly indicates a presence, quantity, and/or activity level of the RTL in contact with the T cell hybrid.

4. The T cell hybrid of claim 4, wherein the T cell hybrid exhibits a Th1 cytokine expression profile.

5. The T cell hybrid of claim 1, wherein the T cell expressing the TCR is a naïve T cell or a central memory T cell (Tcm).

6. The T cell hybrid of claim 1, wherein the T cell expressing the TCR is CD45+.

7. (canceled)

8. (canceled)

9. The T cell hybrid of claim 1, wherein T cell expressing the TCR is CD62L+.

10. The T cell hybrid of claim 1, wherein the T cell expressing the TCR is CD27+.

11. The T cell hybrid of claim 1, wherein T cell expressing the TCR is CD49d−.

12. The T cell hybrid of claim 1, wherein the T cell expressing the TCR is CCR7+.

13. The T cell hybrid of claim 1, wherein the T cell expressing the TCR is LIGHT−.

14-22. (canceled)

23. The T cell hybrid of claim 22, wherein the T cell expressing the TCR is transgenic for a HLA-DR, HLA-DP, or HLA-DQ genotype.

24. The T cell hybrid of claim 23, wherein the T cell expressing the TCR is transgenic for a mutant HLA-DR, HLA-DP, or HLA-DQ isotype that contributes to a mammalian immune disorder.

25-27. (canceled)

28. A mammalian T cell hybrid comprising a fusion product of a mammalian T cell expressing a T cell receptor (TCR) and a mammalian fusion partner cell to yield a clonal T cell hybrid, wherein said T cell hybrid exhibits an antigen (Ag)-specific, TCR-mediated proliferative response following contact of the T cell hybrid with a cognate Ag and an allogeneic Ag-presenting cell (APC), wherein said T cell expressing the TCR is transgenic for a mutant HLA-DR, HLA-DP, or HLA-DO isotype that contributes to a mammalian immune disorder, and wherein said mutant HLA-DR, HLA-DP, or HLA-DQ isotype is selected from a mutant HLA-DR1, HLA-DR2, HLA-DR3, HLA-DR4, HLA-DP2, HLA-DQ6, or HLA-DQ8 isotype.

29. The T cell hybrid of claim 28, wherein the mutant HLA-DR, HLA-DP, or HLA-DQ isotype is selected from the group consisting of DRB1*0101; DRB1*0301, DRB1*0401, DRB1*0405, DRB1*1501, DRB4*0101, DRB5*0101, DPA1*0101, DPA1*0103, DPB1*0201, DPB1*0401, DQA1*0102, DQA1*0501, DQB1*0201, DQB1*0301, DQB1*0302, DQB1*0602, and DQB1*0604.

30-41. (canceled)

42. The T cell hybrid of claim 1, wherein said cognate Ag comprises an immunodominant T cell epitope associated with a mammalian autoimmune disease, inflammatory disorder, allergic condition, cutaneous immune disorder, transplant rejection condition, or graft versus host disease (GVHD).

43. (canceled)

44. The T cell hybrid of claim 42, wherein the T cell epitope is associated with a mammalian autoimmune disease selected from the group consisting of multiple sclerosis (MS), rheumatoid arthritis (RA), insulin-dependent diabetes mellitus (IDDM), chronic beryllium disease, autoimmune uveitis, sarcoidosis, systemic lupus erythromatosis, myasthenia gravis, and celiac disease.

45. The T cell hybrid of claim 44, wherein the autoimmune disease is MS, and wherein the immunodominant epitope is selected from the group consisting of human (hu)-myelin oligodendrocyte protein (MOG)-1-22, huMOG-35-55, huMOG-huMOG-1-22, huMOG-34-54, huMOG-63-87, huMOG-64-96, huMOG-92-106, murine (mu)-MOG-1-30, muMOG-35-55, muMOG-8 1-110, muMOG-91-110, rat (rt)-MOG-1-20, rtMOG-35-55, rtMOG-74-90, guinea pig (Gp)-myelin basic protein (MBP)-72-89, rt-MBP-72-89, hu-MBP-85-99, hu-MBP-86-99, hu-MBP-87-99, PLP-139-151, and hu type II collagen (CII)-259-273.

46-58. (canceled)

59. The T cell hybrid of claim 1, wherein the mammalian T cell expressing the TCR is isolated from a mammalian subject following immunization of the subject with a cognate Ag and prior to differentiation of said T cell to express one or more markers expressed by an effector memory T cell (Tem) and not expressed at detectable levels by naïve T lymphocytes and/or central memory T cells (Tcm).

60-66. (canceled)

67. The T cell hybrid of claim 1, wherein the T cell hybrid has the characteristics of hybrid cells deposited under ATCC accession number: PTA-6082.

68-97. (canceled)
